(19) United States
(12) Patent Application Publication
(10) Pub. No.: US 2025/0033058 A1
(43) Pub. Date: Jan. 30, 2025

(54) ASSAY FOR CONTROLLING ASSAY PROCESSES IN A SAMPLE-TO-ANSWER DEVICE AND METHOD OF USE THEREOF

(71) Applicant: NOVEL MICRODEVICES, INC., Baltimore, MD (US)

(72) Inventors: Rohan Pais, Baltimore, MD (US); Andrea Pais, Baltimore, MD (US); David Campbell, Bel Air, MD (US); Andrew Grzanka, Cockeysville, MD (US); Sean Zakielarz, Fallston, MD (US); Trevorne Nisbett, Rosedale, MD (US); Hamdi Joda, Baltimore, MD (US)

(73) Assignee: NOVEL MICRODEVICES, INC., Baltimore, MD (US)

(21) Appl. No.: 18/706,404

(22) PCT Filed: Nov. 1, 2022
(86) PCT No.: PCT/US2022/048636

§ 371 Date: Jul. 14, 2024

Related U.S. Application Data
(60) Provisional application No. 63/274,507, filed on Nov. 1, 2021.
(60) Provisional application No. 63/274,510, filed on Nov. 1, 2021.

(51) Int. Cl. B01L 7/00; B01L 3/00

(52) U.S. Cl. CPC B01L 7/52; B01L 3/502738; B01L 3/502761; B01L 2200/02; B01L 2200/0647; B01L 2200/16; B01L 2300/0663; B01L 2300/12; B01L 2300/1811; B01L 2300/1894; B01L 2400/043; B01L 2400/0605

(57) ABSTRACT

Disclosed herein is an apparatus for controlling assay processes performed in a microfluidic cartridge used in sample-to-answer and point-of-care diagnostic instruments. The apparatus controls reagent dispensing into a microfluidic cartridge, magnetic bead based movement of analytes within a microfluidic cartridge, as well as nucleic acid sample preparation, amplification, and detection processes.

### CROSS REFERENCE TO RELATED APPLICATIONS

This application is a 35 U.S.C. § 371 national phase non-provisional application of PCT Application Serial No. PCT/US2022/048636 filed on Nov. 1, 2022, which claims priority to U.S. Provisional Application Ser. Nos. 63/274,507 and 63/274,510, each of which were filed Nov. 1, 2021, the disclosures of which are incorporated by reference as if fully set forth herein.

### TECHNICAL FIELD

The general technical field is point-of-care sample-to answer devices that perform assays in microfluidic cartridges and components thereof that are used to control the sequential dispensing of reagents and other assay steps performed by point-of-care sample-to-answer devices.

### BACKGROUND

Point of Care (POC) devices can bring rapid molecular diagnostic testing at the site of patient care. Such sample-to-answer devices and associated procedures commonly utilize molecular amplification techniques like PCR or isothermal amplification, which require control over the sequence of reagent dispensing and other assay parameters.

Typically, liquid reagents are stored in a reagent blister on a microfluidic cartridge with an opening to allow the liquid to flow into the cartridge. The opening is sealed with a rupturable membrane or layer. Ordinarily, the rupturable membrane must be torn or ruptured and the blister crushed to dispense liquid reagent into the cartridge.

Crushing reagent blisters to dispense reagent requires force delivered by structures configured to deform the reagent blister under pressure. This operation is commonly powered by individual geared motors controlling each actuating structure. However, certain complex cartridge designs e.g., with multiple reagents to dispense from multiple reagent blisters sometimes according to a predetermined dispensing sequence, require increased instrument complexity including, for example, increasing the number of motors and drive assemblies required. This increases the size, cost, and portability of the instrument.

Assays performed on sample-to-answer devices often perform molecular amplification on a microfluidic cartridge depending on the assay. Most of these technologies use molecular amplification techniques like PCR or isothermal amplification, which rely on heating (or in the case of PCR, thermal cycling) of the reaction for the amplification.

Most sample-to-answer devices utilize resistive based heaters. In a resistive heat method, an electric current is passed through a resistor element. This current is converted into heat at the resistor that is used to heat the amplification reaction. One issue with this method is the need for precise contact between the reaction chamber on the microfluidic cartridge and resistive heater element. Any loss of contact can lead to poor heat conduction into the amplification chamber and hence a failed amplification. To overcome this issue, few technologies on the market implant the resistive heater elements as part of their disposable cartridge. Alternate approaches involve adding pressure to the amplification well causing it to bulge outwards and improve contact with a heater element.

Another challenge with the contact-based approach is temperature control. In such a setup the temperature sensor is placed on the heater element (instead of the fluid). This approach needs a predictive algorithm to determine the temperature of the fluid in the amplification chamber that is based on the heater element temperature and the ambient temperature. This could lead to inaccurate temperature control.

PCR thermal cycling technologies use “heat zones” whereby PCR reagents are moved between the heat zones during the thermal cycling process. While this is a rapid technique, it requires pumps and accurate fluid movement controls to ensure the temperatures are held for the appropriate cycle times.

### SUMMARY

In accordance with the present invention, various embodiments of the apparatus for controlling assay processes performed in a microfluidic cartridge are disclosed.

In one embodiment, an apparatus for controlling assay processes in a microfluidic cartridge is provided. In some embodiments, the apparatus can be powered by a single motor. In one embodiment, the single motor can be a stepper motor, a servo motor, or a gear motor. In another embodiment, the single motor contains one gear and is configured to operate at one speed.

In another embodiment, the microfluidic cartridge can contain one or more reagent filled blisters, a fluidic channel, and one or more wells or chambers. In another embodiment, the microfluidic cartridge can contain a lateral flow strip. In yet another embodiment, the microfluidic cartridge can contain one or more flow through blisters. The one or more flow through blisters can contain an inlet valve and an outlet valve. In yet another embodiment, the one or more reagent filled blisters comprise crush blisters.

The apparatus can further include a drive belt assembly. In one embodiment, the drive belt assembly can contain a single drive belt. In another embodiment, the drive belt assembly can contain one axle extending from the single motor.

The apparatus can further include a clutch assembly comprising one or more clutches configured to rotate about a central axis of rotation. In some embodiments, each clutch can contain a drive shaft centrally positioned within each of the one or more clutches on the central axis of rotation, and wherein rotation of said one or more clutches is driven by said drive belt assembly. In one embodiment, the clutch assembly contains a plurality of clutches. In another embodiment, the clutch assembly can contain three clutches. In other embodiments, the one or more clutches are electromagnetic clutches. In some embodiments, a single axle extending from the single motor is affixed to only one of the plurality of clutches to power rotation of the drive belt assembly.

The apparatus can further include an actuation mechanism comprising one or more actuation elements mounted to said drive shaft configured to engage the microfluidic cartridge and actuate one or more assay processes. In one embodiment, the one or more actuation elements can comprise one or more press plates. In another embodiment, the one or more press plates can contain one or more protrusions configured for physical engagement with said microfluidic cartridge. In some embodiments, the one or more protrusions comprise a first shape configured to engage and open said inlet valve and outlet valve on the one or more flow through blisters. In other embodiments, the one or more protrusions comprise a second shape configured to deform said one or more crush blisters to force said reagent out of said crush blister. In another embodiment, the drive shaft can contain a threaded portion for mounting said one or more actuation elements.

The apparatus can further include a series of spatially arranged permanent magnets positioned proximate the microfluidic cartridge. In some embodiments, the microfluidic cartridge can contain metal particles (e.g., metal beads) for binding to target analytes and moving the target analytes through the microfluidic cartridge. In other embodiments, the permanent magnets can be configured to move the metal particles through the microfluidic cartridge by magnetic force. In one embodiment, the spatially arranged permanent magnets can be affixed to a rotating wheel positioned adjacent to and substantially coplanar with the microfluidic cartridge.

In another embodiment, a method for controlling assay processes in a microfluidic cartridge is provided. The method can include the step of providing an apparatus configured to be programmed with one or more sequence files corresponding to one or more assays. The apparatus can further include the following components: a single motor; a drive belt assembly comprising a single drive belt; a clutch assembly comprising a plurality of clutches configured to rotate about a central axis of rotation, each clutch comprising a drive shaft centrally positioned within each of the one or more clutches on the central axis of rotation, and wherein rotation of said plurality of clutches is driven by said drive belt assembly; and an actuation mechanism comprising one or more actuation elements mounted to said drive shaft configured to engage the microfluidic cartridge and actuate one or more assay processes. In some embodiments, the method can include the step of programming the apparatus with one or more sequence files corresponding to the one or more assays. In another embodiment, the method can include the additional steps of inserting the microfluidic cartridge into said apparatus and initiating performance of the assay using the apparatus. In some embodiments, the one or more assay processes comprises polymerase chain reaction, magnetic bead based movement of a target analyte through said microfluidic cartridge, and/or lateral flow strip detection. In some embodiments, the detection procedure can be any of lateral flow strip detection, real time optical florescence detection, optical microarray detection, and electrochemical detection.

In one embodiment, the apparatus monitors and controls reaction temperatures, for example, in polymerase chain reaction. Thus, the apparatus used can include a microfluidic cartridge, an actuator plate, and a temperature control unit comprising a heating element, a heat dissipating element, and a temperature sensor. In some embodiments, the temperature sensor comprises an IR temperature sensor.

In another embodiment, the microfluidic cartridge comprises an amplification chamber wherein an assay reaction, such as PCR, is performed. In another embodiment, the heating element comprises an inductive coil element. In yet another embodiment, the inductive coil element comprises a bifilar coil. In yet another embodiment, the heating element can be mounted to a rotating wheel that can move (e.g., rotate) with respect to the cartridge. This movement will present the heating element in proximity to the amplification well when heating is required and remove the heating element during cooling to facilitate rapid heating and cooling. In a further embodiment, the heating element and said amplification chamber are not in physical contact leaving a vacant gap therebetween.

In some embodiments, the heat dissipating element can also be mounted to the rotating wheel. In another embodiment, the heat dissipating element comprises a heat sink. In yet another embodiment, the heat sink is comprised of aluminum, ferrous metals, copper (combinations and alloys of the same), carbon-derived materials in combination with aluminum, and/or natural graphite composite materials.

In yet another embodiment, the heat dissipating elements performance can further be enhanced using fans and/or a water pump to introduce convective cooling either with air or a coolant fluid. In a further embodiment, a thermoelectric cooler (TEC) with or without the combination of a fan/fans and heat sinks can be utilized to enhance the performance of the heat dissipating element.

In some embodiments, the temperature control unit further comprises a heat storage target. In another embodiment, the heat storage target is positioned inside said amplification chamber or in contact with the wall of the amplification chamber to facilitate heat transfer into the fluid contained within the amplification chamber. In another embodiment, the heat storage target is positioned inside the amplification chamber so that the reagents being amplified is in direct contact with the front face and/or the back face(s) of the heat storage target. In yet another embodiment, the heat storage target forms a pocket to envelop the reagents being amplified. This is accomplished by having a recess within the target and allowing the reagents to flow within said recess. This in turn increases the surface area in contact with the fluid, allowing for faster heat transfer. In yet another embodiment, the heat storage target may comprise of fins that will increase the surface area of contact between the target and fluid being heated. In yet another embodiment, the heat storage target is comprised of metal. In some embodiments, the metal is aluminum, aluminum alloys, ferrous metals and their alloys, copper, and or combinations thereof. In yet another embodiment, the metal target may be coated with a polymer to modify its thermal inertial properties. In a further embodiment, the heat storage target is comprised of a material comprising predetermined thermal inertia properties. In another embodiment, the heat storage target material comprises high thermal inertia properties. The heat storage target may be bonded in the amplification chamber with an adhesive or welded to the plastic substrate of the microfluidic cartridge. In yet another embodiment, the heat storage target is suspended inside the amplification chamber. In some embodiments, the temperature control unit is capable heating and cooling rates of said amplification reagent of up to about 15° C. per second. In yet another embodiment, the device is capable of reaction speeds of up to about 40 cycles of PCR in under 15 mins.

In one embodiment, the apparatus is incorporated into a sample-to-answer and/or point-of-care device for carrying out sample-to-answer diagnostic assays. Thus, in one embodiment, a sample-to-answer and/or point-of-care device for the performance of diagnostic assays comprising the apparatus for controlling assay processes in a microfluidic cartridge is contemplated.

### BRIEF DESCRIPTION OF THE FIGURES

Having thus described the presently disclosed subject matter in general terms, reference will now be made to the accompanying Figures which disclose representative embodiments of the invention.

FIG. 1 shows a side perspective of an embodiment of the overall apparatus.

FIG. 2 shows a cross-sectional view of an embodiment of the clutch system.

FIG. 3 shows an embodiment of a clutch assembly, actuator elements, and belt drive assembly.

FIG. 4A-B shows an embodiment of various structural components of the actuator mechanism with three actuator elements.

FIG. 5A-B shows an embodiment of a first actuator element and microfluidic cartridge.

FIG. 6A-B shows an embodiment of a second actuator element and microfluidic cartridge.

FIG. 7A-B shows an embodiment of a third actuator element and microfluidic cartridge.

FIG. 8A-C shows an embodiment of an electromagnetic clutch.

FIG. 9A-B shows an embodiment of the rotating wheel with mounted permanent magnets and a microfluidic cartridge.

FIG. 10 shows an embodiment of a microfluidic cartridge with an amplification chamber and

heat storage target contained therein.

FIG. 11A-C shows an embodiment of a rotating with mounted permanent magnets, heating element, and heat sink, interfacing with a microfluidic cartridge.

FIG. 12A-C shows an embodiment of a rotating wheel with mounted permanent magnets, heating element, and a heat sink with a thermoelectric cooler (TEC) and heat spreader.

FIG. 13 shows an embodiment of a heating element, microfluidic cartridge, amplification chamber, heat storage target, and temperature sensor configuration.

FIG. 14A-E shows embodiments of heat storage target positioning and shape within an amplification chamber.

FIG. 15 shows an embodiment depicting three target temperature regions in a segmented fluidic channel (valved) and the movement of reagent through the channel from one region to the next.

FIG. 16 shows an embodiment depicting three target temperature regions in a continuous fluidic channel (not valved) on a disk and movement of reagent through the channel from one region to the next.

FIG. 17 shows lateral flow strip and electrophoresis images showing positive CT and NG targets according to the example.

FIG. 18A-B shows a rotating wheel alone and interfaced with microfluidic cartridge according to the example.

FIG. 19A-C shows a rotating wheel and a temperature sensor interfaced with a microfluidic cartridge according to the example.

FIG. 20 shows lateral flow strip image showing positive CT-11 following assay performed in the example.

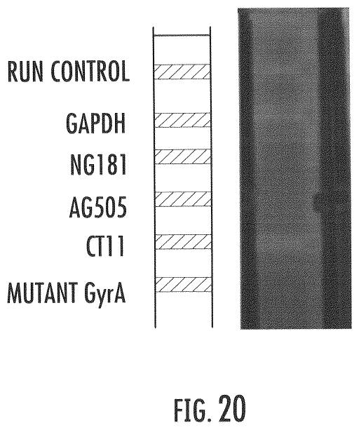

### DETAILED DESCRIPTION

The presently disclosed subject matter now will be described more fully hereinafter with reference to the accompanying Figures, in which some, but not all embodiments of the presently disclosed subject matter are shown. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### Reagent Dispensing Control

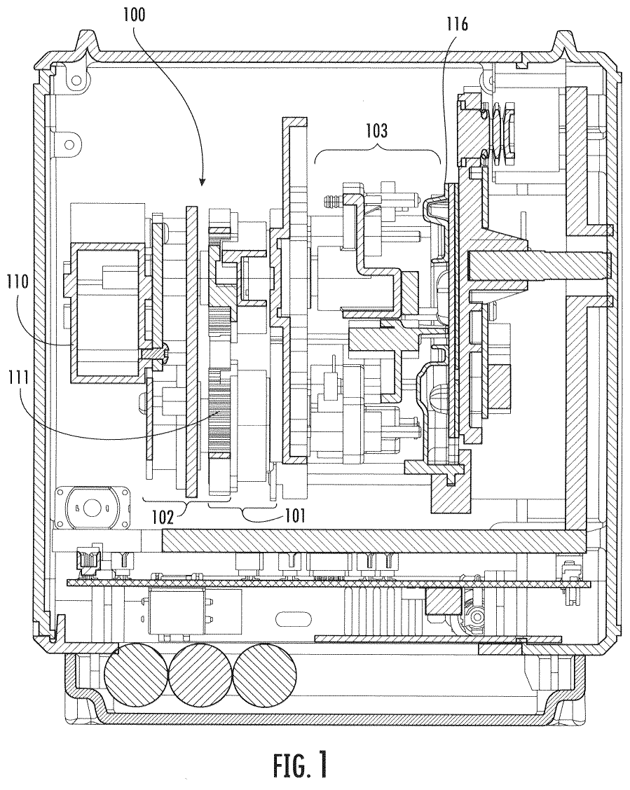

Referring to FIG. 1, an embodiment of the apparatus 100 that is used for the controlled sequential dispensing of reagents, generally can include a clutch assembly (shown generally at 101), a belt drive assembly (shown generally at 102), and an actuator mechanism (shown generally at 103). The apparatus 100 is configured to sequentially dispense reagents from one or more reagent dispensing units positioned on or in a microfluidic cartridge in a predetermined and controlled manner dictated, in large part, by the specific procedural requirements of the diagnostic assay being performed.

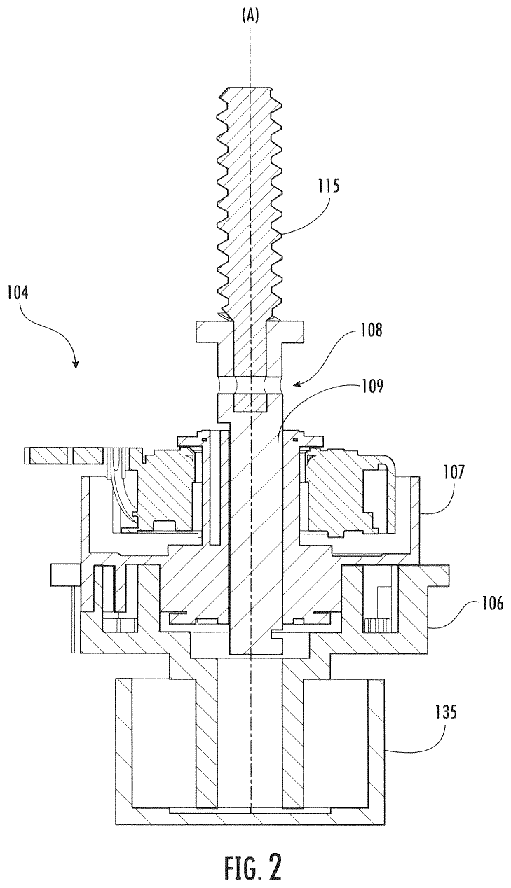

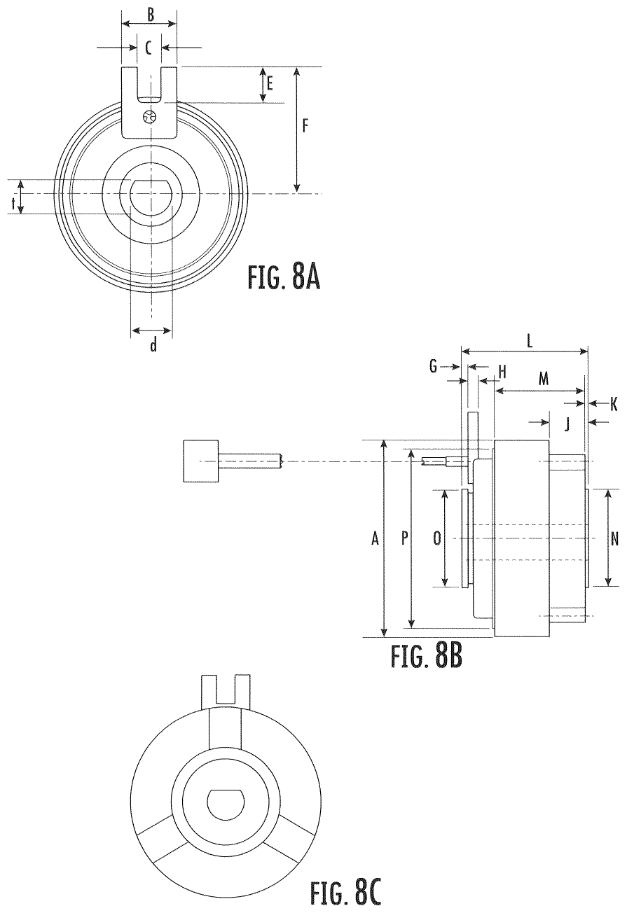

Referring to FIG. 2, an embodiment of a clutch assembly 101 is illustrated. While other clutch assembly designs may be used, the clutch assembly 101 shown in FIGS. 1-3 is an electromagnetic clutch assembly. In some embodiments, electromagnetic clutch assemblies, such as those manufactured and sold by Ogura Industrial Corp., 100 Randolph Road, Somerset, NJ, 08873 (https://ogura-clutch.com) can be used. Particularly, in some embodiments, clutch assemblies falling within the MIC Electromagnetic Micro Clutch product line, manufactured by Ogura Industrial Corp can be used. Other known manufacturers of micro EM clutch assemblies include, but are not limited to, Miki Pulley, US, 13200 Sixth Avenue North, Plymouth, MN, 55441; and Chain Tail Co., Ltd., No. 305, Lane 460, Sec.1, Hsinan Rd., Wu-jih Dist., Taichung City, Taiwan, 41462. Other examples of electromagnetic clutch assemblies are disclosed in U.S. Pat. Nos. 7,325,664B2; 6,997,294 B2; 7,040,374 B2; 8,235,196 B2; 6,837,351 B2; 7,581,628 B2; and 6,823,974 B2. Table 1 below comprise exemplary technical specifications for an electromagnetic clutch with 2.5, 3.5, and/or 5 torque parameters. The referenced letters, for example “d” and “t” for the Bore, are shown in FIG. 8A-C.

| MIC | | 2.5T | 3.5T | 5T |
|---|---|---|---|---|
| Static Torque [in-lbs/N-m] | | 2.2/0.25 | 3/0.34 | 7.1/0.8 |
| Coil (20° C.) | Voltage [DC-V] | 24 | | |
| | Resistance [Ω] | 225.6 | 240 | 202.6 |
| | Wattage [W] | 2.6 | 2.4 | 2.8 |
| Armature* | Engage Time [ms max] | 20 | 15 | 15 |
| | Stop Time [ms max] | 20 | 20 | 25 |

| Bore [mm] | d | 6 | 8 | 6 | 8 | 6 | 8 |
|---|---|---|---|---|---|---|---|
| | t | 5 | 6.5 | 5 | 6.5 | 5 | 6.5 |

| Dimensions [mm] | A | 28 | 31.5 | 33 |
|---|---|---|---|---|
| | B | 8 | 8 | 9 |
| | C | 3.2 | 3.2 | 3.2 |
| | E | 5 | 4.5 | 6 |
| | F | 18 | 21 | 23 |
| | G | 0.8 | 1.4 | 0.7 |
| | H | 1.5 | 1.5 | 1.8 |
| | J | 5 | 5 | 5 |
| | K | 0.5 | 0.5 | 0.5 |
| | L | 18 | 18 | 22 |
| | M | 13 | 13.2 | 17 |
| | N | 14 | 15 | 16 |
| | O | 14 | 15 | 16 |
| | P | 25.5 | 26.9 | 28.6 |

| | | | |
|---|---|---|---|
| Weight [lbs/kg] | 0.07/0.03 | 0.07/0.03 | 0.13/0.06 |

In one embodiment, the clutch assembly 101 can contain one or more clutches 104 driven by a power source. In some embodiments, the clutch assembly 101 can be electromagnetically controlled. A single clutch 104 is shown in FIG. 2. As illustrated in FIG. 2, a clutch 104 can comprise a hub and rotor assembly 105 comprising a rotor 106 and a hub 107. Rotation of rotor 106 can be driven by a drive belt assembly 102. In some embodiments, drive belt assembly 102 can contain a drive belt (e.g., toothed drive belt) positioned about and engaged to the exterior output surface of the rotor 106 rotating the rotor 106 (or another output structure 135 connected to the rotor 106) as long as the drive belt assembly 102 is receiving power. In another embodiment, hub 107 is also rotated when the rotor 106 and hub 107 are mechanically engaged. In related embodiments, hub 107 can be shifted via electrical signal from an engaged (active or rotating) to a disengaged (inactive or stationary) state. In some embodiments, when the clutch 104 is in an un-potentiated or neutral state (i.e., no coupled rotation of rotor and hub), rotor 106 and hub 107 can be slidably engaged to one another or completely disengaged such that the rotor 106 can rotate freely under power while hub 107 remains stationary. A bearing assembly may be used for sliding engagement. In one embodiment, an electrical signal is sent to hub 107 resulting in magnetically driven frictional engagement of structural components of hub 107 with rotor 106 thereby producing coupled rotation of rotor 106 and hub 107. In some embodiments, clutch 104 can house a drive shaft or rod 108 in a drive shaft housing 109 centrally positioned within the clutch 104 along axis (A). As shown in FIG. 2, drive shaft 108 can contain a threaded portion 115 for mounting of, for example, one or more actuator elements as described below. Drive shaft housing 109 extends along axis (A) through both rotor 106 and hub 107.

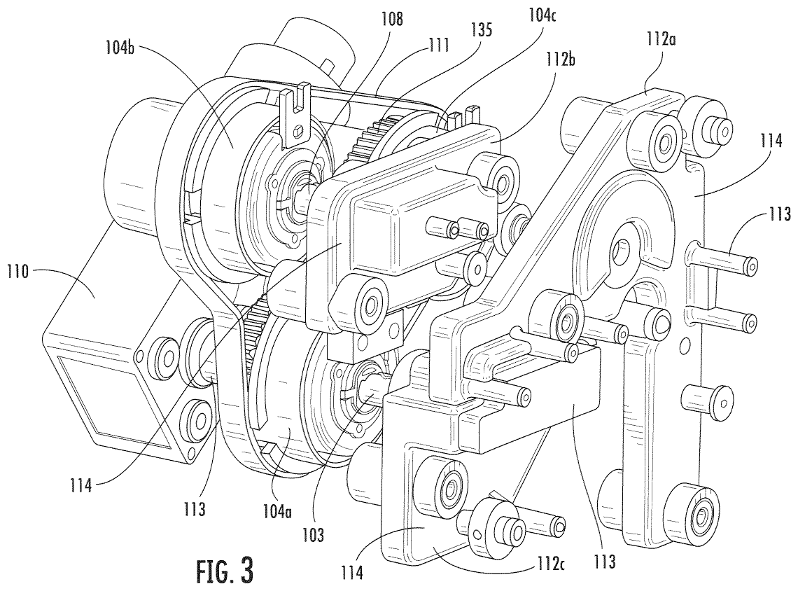

FIG. 1 illustrates an embodiment of the apparatus in general. As mentioned above, the apparatus can include a clutch assembly (shown generally at 101), a belt drive assembly (shown generally at 102), and an actuator mechanism (shown generally at 103). In the embodiment illustrated in FIG. 1, a motor 110 is used to power a drive belt 111 via drive axle 113 (best shown in FIG. 3). In some embodiments, the motor 110 can be a servo motor, a stepper motor, a single phase gear motor, a DC motor, an AC induction motor, and the like. In another embodiment, the motor 110 can contain a single gear and operate at a single speed. Drive belt 111 is shown to be mounted to and wrapping around two output structures 135, which are engaged to rotor elements 106a-b shown in the figure. Drive belt 111 can be mounted to output structure 135 that is configured to engage rotor 106. Alternatively, drive belt 111 can be mounted to rotors 106. In some embodiments, the motor 110 can contain a single gear. One of ordinary skill in the art will appreciate that additional pulleys and/or rotating components, such as the output structure described above configured for rotor 106 engagement, can be added in line with the motor 110. For example, if tighter control over the drive belt's angular position is desired, a servo motor may be utilized for closed loop control of the angular position of the belt as well as detect belt slippage on rotors. This arrangement may be useful when other components besides the clutches are utilizing the drive belt's rotation.

With continued reference to FIG. 1, drive belt 111 can comprise a rubber drive belt. In other embodiments, the rubber drive belt can be toothed for less slippage. In another embodiment, a drive chain can be used for even less slippage. When a drive chain is used, rotor 106 (or another output structure 135 configured to engage rotor 106) can include a gear or sprocket for chain engagement. As described above, control of hub 107 and rotor 106 engagement/disengagement is by electrical signal and magnet activation. When hub 107 is engaged to rotor 106, the drive belt 111 rotates the clutch assembly, including both hub 107 and rotor 106, which can be described as coupled rotation. Coupled rotation of the hub and rotor assembly causes linear movement of the drive shaft 108 (forward or backward) through the clutch components.

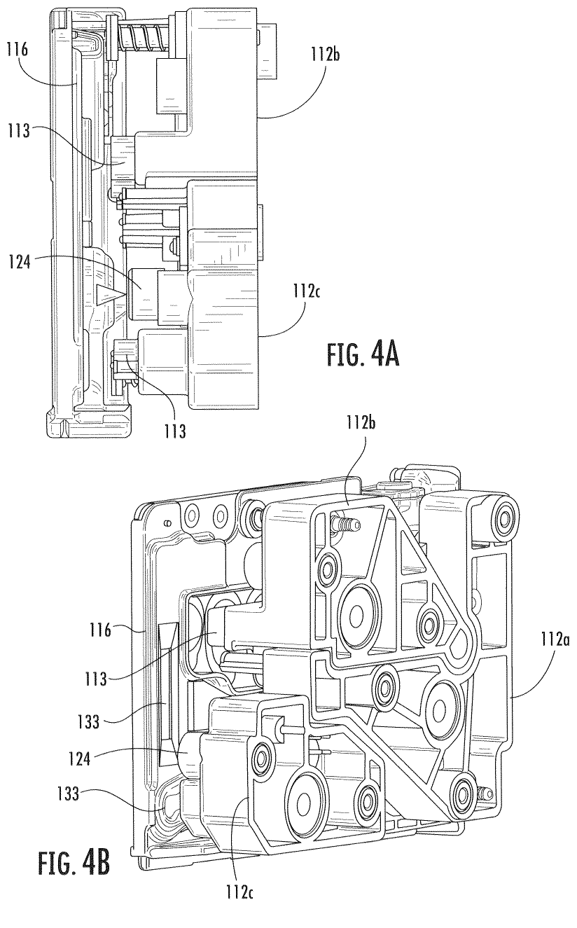

Referring to the embodiment illustrated in FIG. 3, one or more drive shafts 108 are engaged to (also, for example, anchored to or set within) one or more actuator elements 112 inside the sample-to-answer diagnostic instrument. In the embodiment shown, there are three clutches 104a-c, three drive shafts 108a-c, and three actuator elements 112a-c (also shown in FIG. 4A-B). It should be noted that the three clutches, drive shafts, and actuator elements are powered by a single motor 110 and a single drive belt assembly 102. In this embodiment, the motor 110 supplies direct power to a first clutch 104a via drive axle 113 causing rotation of the clutch 104a. Rotation of second clutch 104b and third clutch 104c is achieved via drive belt 111 that wraps outputs of rotors 106a-c.

It should be noted that single motor potentiated rotation of a plurality of clutches producing controlled actuation of a plurality of actuation elements greatly reduces the size, complexity, cost, and power needs/consumption of the sample-to-answer diagnostic instrument. Such a design results in a more compact and portable instrument without sacrificing diagnostic assay speed, accuracy, and efficiency. Thus, precision control over a predetermined reagent dispensing sequence (adjustable depending on the diagnostic assay) from a single microfluidic cartridge 116 using a single power source provides a technical advantage over known sample-to-answer devices.

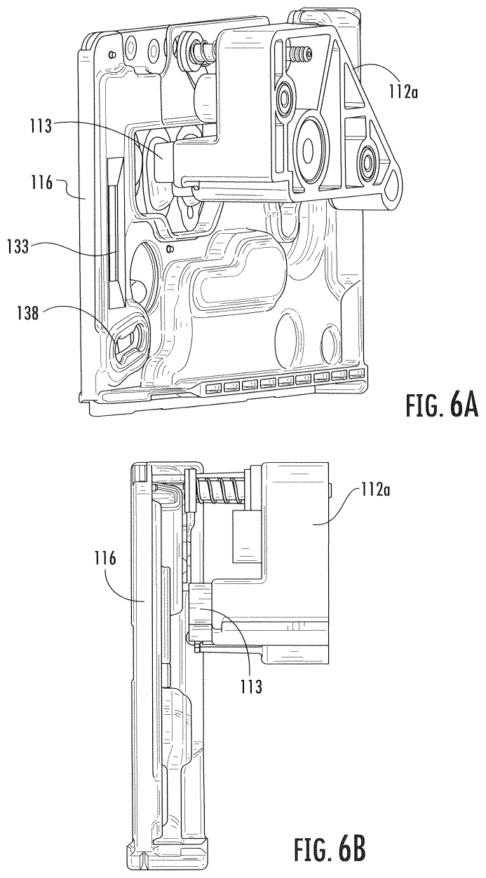

Referring to the embodiment illustrated in FIGS. 3-6, the one or more actuation elements 112, in some embodiments, are press plates with one or more protrusions 113 configured to contact the reagent dispensing units (RDUs) on the microfluidic cartridge as the press plates move toward the cartridge 116 surface via drive shaft 108. As shown in FIGS. 4-6, each of the three press plates 112a-c can contain one or more protrusions 113 on a press plate front face 114 configured to apply pressure to specific structural features of the microfluidic cartridge 116. The one or more protrusions can be of uniform size and shape, or they can be of variable size and shape. Indeed, in some embodiments, the one or more protrusions on the press plates are replaceable or interchangeable i.e., can be removed and replaced with other protrusions (of different size and/or shape) depending on the microfluidic cartridge blister configuration and/or assay procedure. Alternatively, the press plates themselves can be detached from the threaded portion 115 of drive shaft 108 and exchanged or replaced with other plates containing a different set of protrusions.

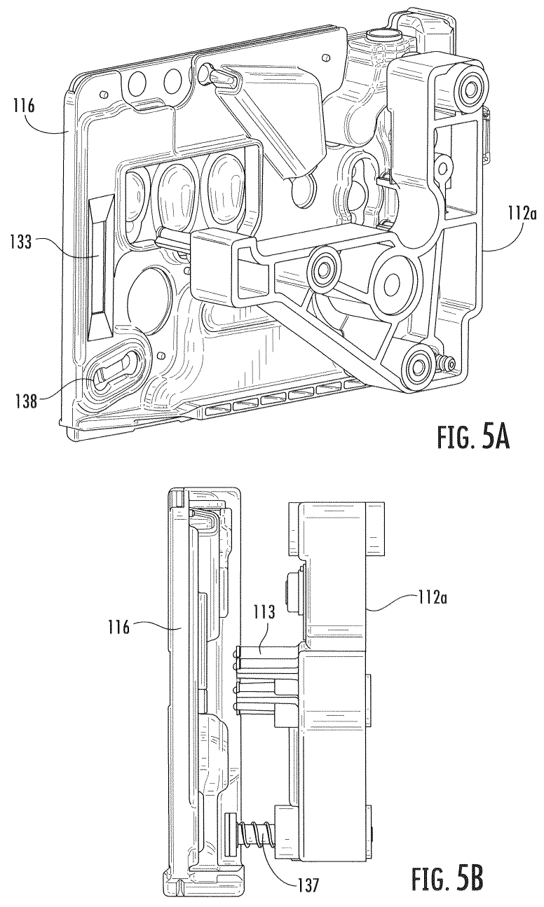

For example, some protrusions 113 are configured (size, shape, and overall design) to engage and open certain inlet and outlet port valves on a flow through blister 138 (shown in FIGS. 4B and 5A) positioned on the cartridge 116. In some embodiments, these protrusions can be elongated column shaped extensions that, for example, apply pressure to bead-containing vessels causing the bead to rupture a foil lidding thereby opening the inlet/outlet valves. Other protrusions 113 are configured to press down upon and crush (or deform) reagent filled blisters to force reagent contents out of the blister and into the microfluidic cartridge 116. In some embodiments, the size and shape of these protrusions depends upon the blister configuration on the cartridge 116.

In this manner, the sample-to-answer instrument can be programmed in a predetermined manner such that each of the one or more actuation elements 112 is actuated forwards or backwards to engage and/or disengage the one or more blisters in the correct sequence needed depending on the diagnostic assay being performed. In this system, the amount of pressure applied to the blister can also be controlled by, for example, controlling the distance traveled by the press plate or the length of the protrusion used. Programmable software driven microfluidic systems have been described in, for example, Mezic et al., US/2009/0038938A1, Microfluidic Central Processing Unit and Microfluidic Systems Architecture.

It should be noted that actuation elements 113 can further contain one or more permanent magnets configured to engage and translocate analyte bound magnetic beads through the fluidic channels of a microfluidic cartridge 116. Magnetic beads with ionizable groups that have a net positive charge at a given pH are used for binding DNA. An example of such as type of magnetic bead is the ChargeSwitch Magnetic bead (Life Technologies, Inc. Carlsbad, Calif.). DNA binds to such a type of magnetic bead in the presence of a binding buffer capable of creating a net positive charge on the bead and DNA is eluted in the presence of an elution buffer capable of creating a net neutral or negative charge on the bead. In this embodiment, ChargeSwitch magnetic beads that cause DNA to bind to them in the presence of a buffer having a pH<5 and elute from them in the presence of a buffer having a pH >8 are used. The magnetic beads are sequentially moved through all the wells on the microfluidic cartridge by methods described in this disclosure. DNA is first allowed to bind to the magnetic beads in the presence of a binding buffer. The beads are then moved in and out of two wells containing wash buffer to wash the impurities from the beads. The impurities are left behind in the wash buffer solution. The washed beads are then moved to a well containing elution buffer where the DNA is eluted from the beads into the elution buffer. The eluted DNA may then be used to hydrate lyophilized reagents for a Nucleic Acid Amplification Test (NAAT). In some embodiments, the magnetic beads may be eluted directly into the amplification well. One or more heaters is turned on as part of the automation sequence to supply the temperatures for amplification.

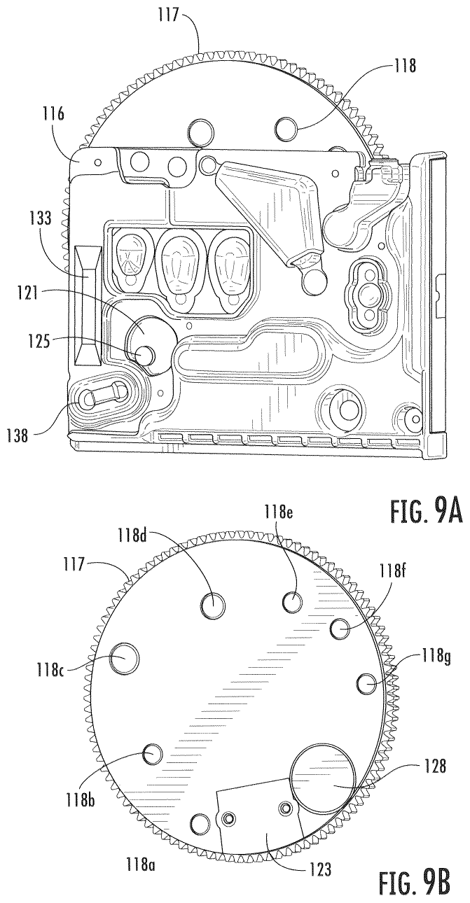

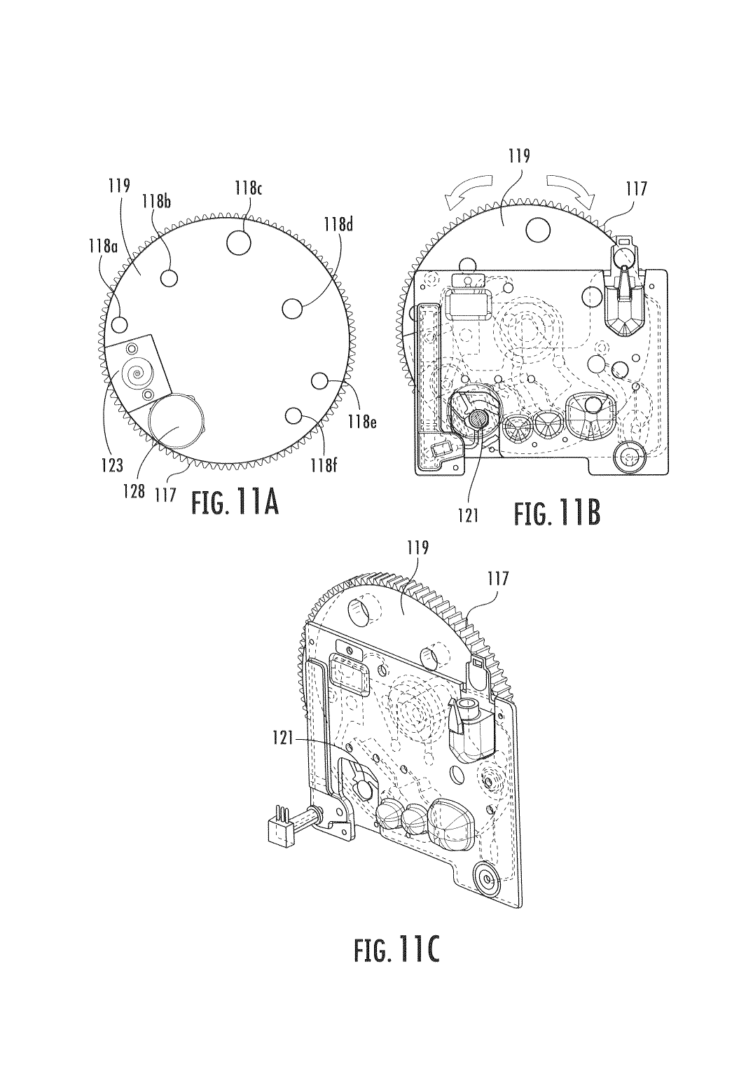

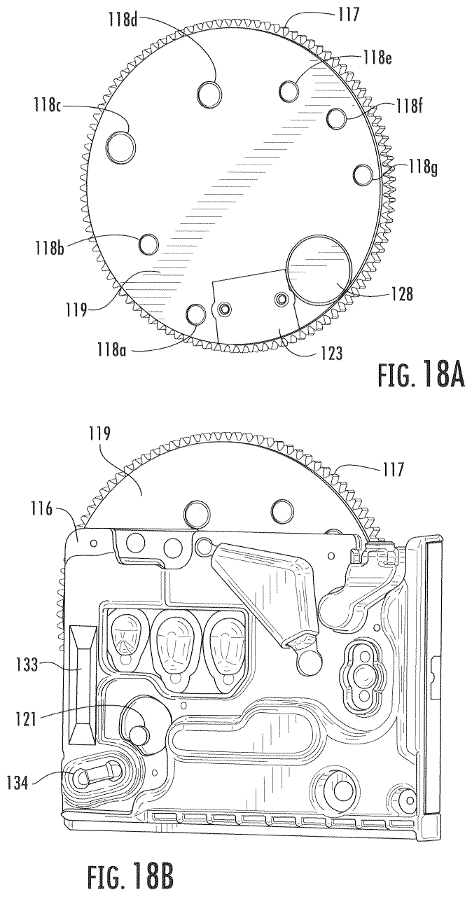

In some embodiments, the actuation elements 112 or the microfluidic cartridge 116 is configured to rotate while remaining substantially coplanar with the actuation element or microfluidic cartridge (depending on which one is able to rotate). Alternatively, as shown in FIG. 11A-C, the spatially arranged one or more permanent magnets can be positioned on a wheel 117 configured to rotate about an axis that is perpendicular to the microfluidic cartridge. Wheel and microfluidic cartridge remain substantially co-planar while wheel 117 rotates with respect to the cartridge. As stated above, wheel 117 can include one or more permanent magnets 118 spatially arranged in a predetermined manner. The embodiment shown in FIGS. 9 and 18, contains seven magnets at specific positions about the face 119 of wheel 117. In this embodiment, the microfluidic cartridge 116 does not rotate, the wheel 117 is positioned on the opposite side of the microfluidic cartridge 116 as the actuation elements 112, and the permanent magnets 118 are positioned on the wheel face 119 closest to the microfluidic cartridge 116. The interaction between wheel 117, magnets 118, and cartridge 116 is explained in greater detail in the Example below.

With continued reference to FIGS. 3-6, press plates 112a-c actuate in a controlled predetermined manner throughout the assay process to move reagents through the cartridge at the correct times. For example, when an assay calls for nucleic acid amplification, press plate 112a can be used to dispense liquid reagent to the wells, plate 112b can be used to fill the remaining volume of the cartridge with a mineral oil, and plate 112c can be used to transfer the amplified product from the primary channel of the cartridge over to the lateral flow strip 133 of the cartridge. In this example, each press plate (and protrusions) can continue to exert pressure on the cartridge until a programmed current limit on the motor is hit which sends a message that the plate is fully extended and in position. The final position of the plates can also be determined with a physical sensor such as an end stop toggle switch or a proximity sensor. In one embodiment, as briefly discussed above and described in greater detail in the below Example, the last steps in the assay can be performed using permanent magnets spatially arranged in predetermined positions on a structure (e.g., rotating wheel 117) that engage magnetic beads bound with nucleic acid molecules and rotation of the microfluidic cartridge. Following elution of the amplified product from the magnetic beads, the amplified product is moved to another station for detection. Detection can be performed using processes such as lateral flow strip detection, real time florescence detection, optical microarray detection, or electrochemical detection.

### Temperature Control-Molecular Amplification (PCR)

The sample-to-answer device also controls and regulates the temperature of a reagent. Even more specifically, the sample-to-answer device can regulate, heat, cool, and/or measure the temperature of reagents used for molecular amplification processes, such as PCR. In one embodiment, the sample to answer device applies a non-contact approach to heating and measuring temperature.

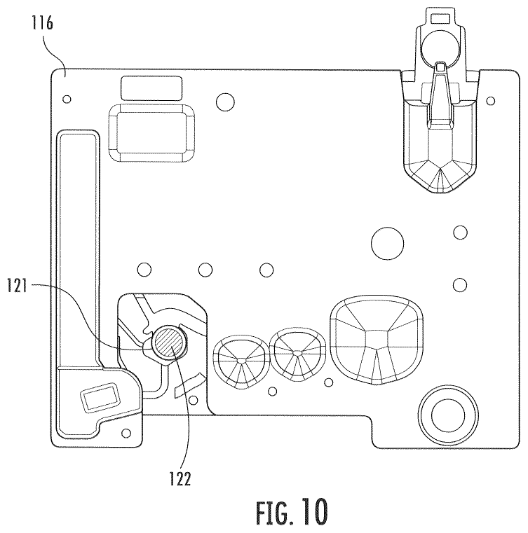

FIG. 10 shows the microfluidic cartridge 116 and an amplification chamber 121 (cross section through the center of the amplification well) positioned within the cartridge 116. In this embodiment, a reagent to be heated is inside the amplification chamber along with a heat storage target 122.

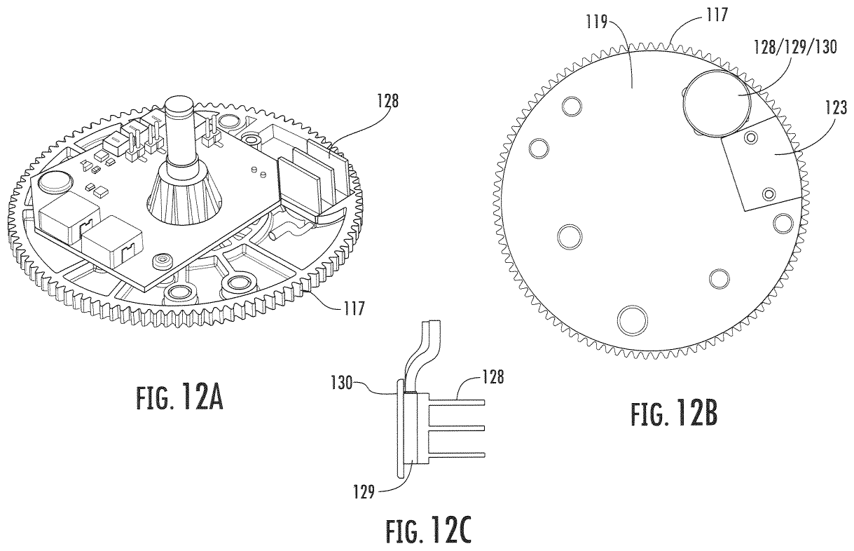

Referring now to FIG. 11, heating and temperature control requires a heating element 123. In some embodiments, the heating element 123 can be an electrical conductor, such as a wire in the shape of a coil, spiral or helix. In another embodiment, the heating element 123 is an inductive coil, such as the embodiment illustrated in FIGS. 11-13. In yet another embodiment, the inductive coil 123 can be an electromagnetic coil, such as a bifilar coil. In one embodiment, an inductive coil 123 is positioned proximate the amplification chamber 121. For example, referring to the embodiment illustrated in FIGS. 11-12, the inductive coil 123 can be positioned on said rotating wheel (actuation wheel) 117 along with the spatially separated permanent magnets 118a-f. FIG. 11A-C illustrates an example of a rotating wheel 117 carrying the inductive coil 123 in relation to the microfluidic cartridge 116 during normal operation.

Alternatively, the heating element can be a resistive heating element comprising a resistor embedding in a block of thermally conducting material such as a metal, metal oxide or metal alloy. The heating element may also be a resistive thin-film heating element or a peltier element. In other embodiments the heating element is a positive temperature coefficient self-regulating element. The heating element may be integrated as part of the microfluidic device and intended as a single use, disposable element. In an additional embodiment, a phase change material may be used to generate the heat energy for lysis and sample transfer. Phase change materials are widely used for a variety of applications requiring thermal energy storage and have been developed for use across a broad range of temperatures (−40° C. to more than 150° C.). Phase change materials are advantageous because they offer high-density energy storage and store heat within a narrow temperature range. Additionally they are inexpensive, non-toxic and do not require electrical energy for generating heat. As such, they are an appealing choice for point-of-care settings and for single-use devices that require heat energy. In an additional embodiment a phase change material contained in a sealed pouch is used to form a jacketed sheath around the sample extraction container. The jacket of phase change material may either be present as part of the microfluidic device or as part of the sample extraction container. The act of connecting the sample extraction container to the microfluidic device works to create a nucleation site that in turn activates the phase change material, causing it to rise in temperature and heat the sample. This may be accomplished by packaging a metal piece in the pouch containing the phase change material, which snaps when the sample extraction container is connected to the microfluidic device. The phase change material may also be activated by an external actuator present on the actuating element in the microfluidic device. In some embodiments, a suitable phase change material may be activated to cool the sample so as to prevent its degradation. In some embodiments, the heating element may serve to heat lyse and transfer the sample to the fluidic well on the microfluidic devices, as well as to run a NAAT on the microfluidic device.

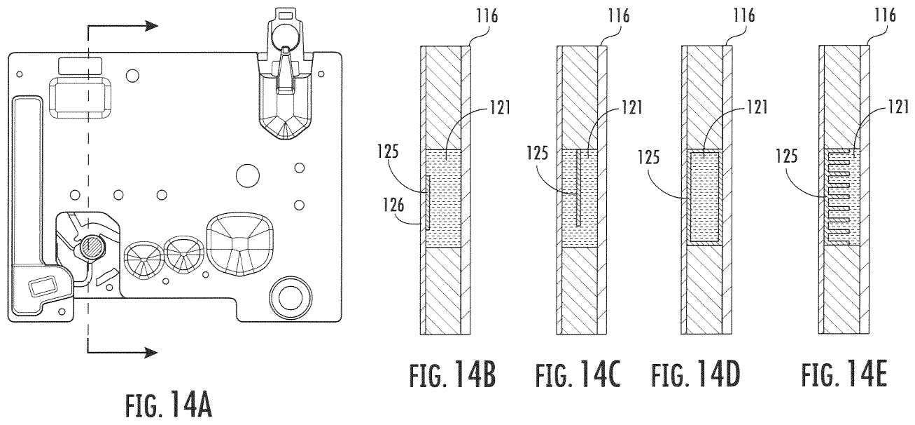

Referring to FIGS. 10 and 14A-E, in some embodiments, the sample-to-answer device can contain a heat storage target 125 positioned inside the amplification chamber 121 of the microfluidic cartridge 116. Various materials may be used for the heat storage target 125 as long as the heat storage target has certain properties such as a proper thermal inertia. In some embodiments, the proper thermal inertia is moderate to low. In some embodiments, the heat storage target is comprised of a metal, for example, aluminum, aluminum alloy, or copper.

As shown in FIG. 14B-E, the heat storage target 125's position within the amplification chamber can be modified or changed. For example, as shown in FIG. 14B, the heat storage target can be adhered to a back surface 126 of the amplification chamber 121. In an alternative embodiment (FIG. 14C), the heat storage target can be centrally positioned within the amplification chamber 121. The heat storage target 125's shape can be modified. For example, as shown in FIG. 14D, the heat storage target 125 can substantially conform to the interior walls of the amplification chamber 121 and enclose the reagent within the chamber. In another embodiment, the heat storage target 125 can be comb shaped (FIG. 14E). The versatility provided by virtue of such modifications permit a variety of assays and modification decisions can be assay dependent. By modifying the size, shape and/or positioning of the heat storage target within the amplification chamber heating can be localized to certain part(s) within the amplification chamber. In some embodiments, heat localization can be controlled by the shape, size/thickness, and thermal mass of the heat storage target. Such a localized heating techniques promote low thermal inertia and hence rapid cooling-highly useful functions for PCR and thermal cycling processes.

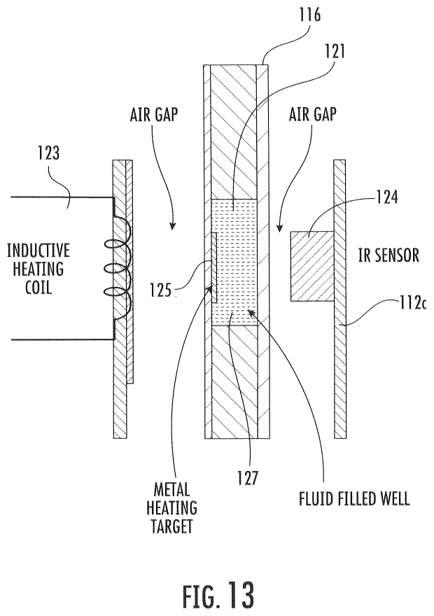

Referring to the embodiment shown in FIG. 13, an oscillating signal can be supplied to the heating element 123 which in turn induces currents in the heat storage target 125 that is contained inside the amplification chamber. This causes the heat storage target 125 to heat up rapidly which in turn heats the amplification reagent 127 that is in contact with the heat storage target 125. It is important to note that, in this embodiment, there is no contact between the heating element 123 and heat storage target 125 inside the amplification chamber. Thus, through such heating strategy, a non-contact method of heating the amplification well on a microfluidic cartridge is realized. Technical advantages of such an approach include rapid heating rates and reliable/repeatable heating of the amplification chamber 121 without the need for precise contact between the heating element 123 and the amplification well 121.

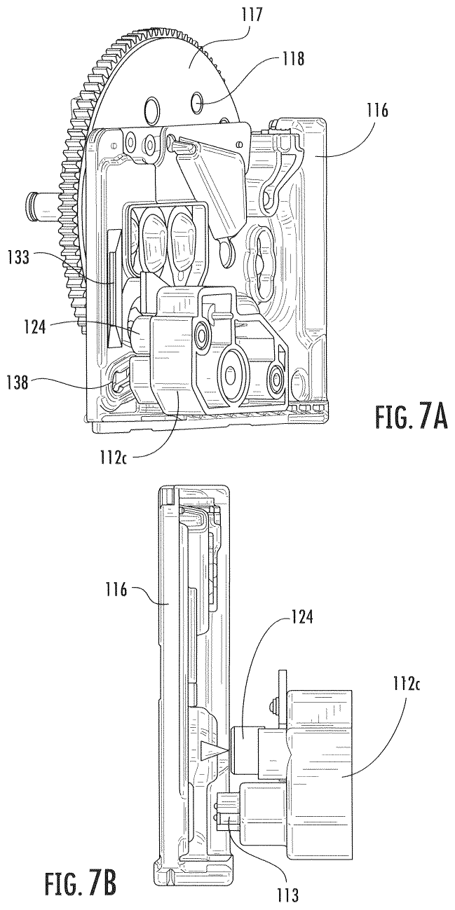

Accurate control of the reagent temperature during thermal cycling is critical for successful polymerase chain reaction and requires accurate temperature monitoring and measurement. Various temperature monitoring techniques are known by those of skill in the art-some more effective than others. In one embodiment, monitoring and measurement can be carried out by one or more sensors or other means that do not contact the measurement target. FIG. 7B illustrates an embodiment of a temperature sensor 124 that can be used to measure the reagent temperature inside the amplification chamber 121. In some embodiments, the temperature sensor 124 can be an infrared (IR) temperature sensor. In other embodiments (see for example, FIG. 7) the temperature sensor is positioned on the opposite side of the microfluidic cartridge from rotating wheel 117 and inductive coil 123. In another embodiment, the temperature sensor 124 is positioned on one of the actuation elements 112 (e.g., 112c) as shown in FIG. 7A-B. In the embodiment shown in FIGS. 7B and 13, the temperature sensor 124 is not in contact with the amplification chamber, heat storage target, or reagent. IR temperature sensors are typically electronic and non-contacting sensors that emit IR radiation. Two types of IR temperature sensors commonly used are standard IR sensors and Quantum IR sensors.

As discussed above, in one embodiment, an infrared (IR) temperature sensor 124 can be positioned proximate the microfluidic cartridge's amplification chamber 121—within its field of view. Amplification chambers 121 can be made of materials that permit IR radiation wave penetration such as, in one example, a thin polycarbonate which is relatively transparent to IR radiation. In this manner, the IR sensor can measure the temperature of the fluid inside the amplification chamber 121 for a “true closed loop” temperature control. In other words, a heating element 123 (e.g., bifilar coil) and temperature sensor 124 positioned on opposite sides of the amplification chamber 121 containing a heat storage target 125 and reaction reagent creates a closed loop temperature control system.

In some embodiments, other temperature sensors may be suitable depending on the system requirements. For example, thermocouple sensors, thermistor sensors, resistance temperature detectors, or a semiconductor based sensor may be used in some embodiments.

In one embodiment, the heating element and temperature sensor can be on either side of the amplification chamber as long as the heating element is proximate to the heat storage target, wherever it is positioned within the amplification chamber, and the temperature sensor is proximate the reagent. In some embodiments, the heat storage target is positioned on the surface of the amplification chamber closest to the heating element, as illustrated in FIG. 13. In other embodiments, the heat storage target is an integral part of the chamber comprising all or a part of the surface. In embodiments where the heat storage target is positioned upon the chamber surface, the thickness of the chamber material below the target may be thinner than other parts of the chamber to promote efficient heating.

Due to the low thermal inertia of the heat storage target 125 material (e.g., metal target), effective temperature control for PCR thermal cycling (either 2 step or 3 step) can be accomplished via single heating element (e.g., inductive coil). The amount of heat produced by the single heating element 123 can itself be controlled to maintain different temperatures in the amplification chamber. In other embodiments, more than one heating element 123 can be used in some embodiments.

Referring to FIGS. 11 and 12, a heating element 123 (e.g., inductive coil) and a structure that acts as a pathway for heat to dissipate away from the cartridge, such as a heat sink 128, are mounted on the rotating wheel 117. Examples of heat sink 128 materials include aluminum, copper (combinations and alloys of the same), carbon-derived materials in combination with aluminum, and natural graphite composite materials. In this embodiment, the rotating wheel 117 is connected to a power source for rotation about its central axis relative to a microfluidic cartridge placed in front of it. In the embodiment shown in FIG. 7, a temperature sensor 124 can be mounted on a structure proximately positioned to the amplification chamber 121 and used to monitor the temperature of the fluid in the cartridge. The temperature sensor 124 shown in this embodiment is mounted to a structure positioned on the side of the microfluidic cartridge (i.e., amplification chamber 121) opposite the rotating wheel 117 which carries the heating element 123 and heat sink 128.

Referring to FIGS. 11 and 12, in some embodiments, the heating element 123 and the heat sink 128 can be spatially oriented (e.g., distance between controlled) on the rotating wheel 117 so that when the wheel is rotated, either the heating element 123 or the heat sink 128 will interface with the amplification chamber 121 of the microfluidic cartridge, but not both simultaneously. When the heating element 123 interfaces with the amplification chamber 121, the heat storage target 125 inside the cartridge is rapidly heated, this in turn, heats the reagent contacting the heat storage target 125. The temperature sensor 124 which is positioned in front of the amplification chamber 121 measures the temperature of the fluid contained therein. In some embodiments, when thermal mass of the heat storage target 125 and the volume of fluid to be heated (locally in front of the target) is small, rapid heat rate of >10 C/s can be achieved.

With continued reference to the embodiment shown in FIG. 11A-C, to rapidly lower the temperature of the fluid, a heat sink 128 can be employed. In this embodiment, the rotating wheel 117 is rotated so the heat sink 128 is now interfaces with the amplification chamber 121. In this embodiment, the heat sink 128 draws out heat from the reagent rapidly. Alternative embodiments shown in FIG. 12A-C, could employ a thermoelectric cooler (TEC) 129 to further improve the cooling rate. Cooling rates of up to about 15 C/s have been observed using the configuration described herein greatly improving PCR speeds. For example, rapid PCR of up to about 40 cycles in under 15 mins can be realized using this novel configuration. In other embodiments, a heat spreader 130 can be used. In some embodiments, the heat sink 128, thermoelectric cooler (TEC), and heat spreader 130 form a sandwich wherein the heat spreader 130 is closest to the amplification chamber 121 and thermoelectric cooler 129 is between the heat sink 128 and heat spreader 130. Heat spreaders transfer energy as heat from a hotter source to a colder heat sink or heat exchanger. The most common type of passive heat spreader is a plate or block of material having high thermal conductivity, such as copper, aluminum, or diamond. Active heat spreaders speed up transfer using an external energy source. Thermoelectric coolers operate by the Peltier effect. Generally, TECs have two sides, and when a DC electric current flows through the device, it brings heat from one side to the other. Ordinarily, the hot side is attached to a heat sink to maintain ambient temperature. Zhao, Dongliang (May 2014). “A review of thermoelectric cooling: Materials, modeling and applications.” Applied Thermal Engineering. 66 (1-2): 15-24. doi: 10.1016/j.applthermaleng.2014.01.074.

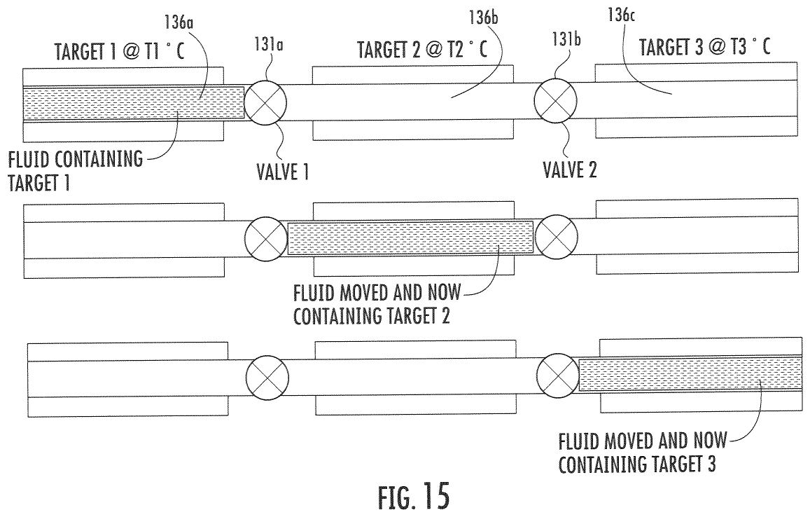

FIG. 15 shows yet another embodiment where a plurality of temperature target regions 136a-c are used (three in this embodiment). A first temperature target region 136a heats the reagent contained in a first amplification chamber 121a (or other reagent well), temperature target region 136b heats the reagent contained in a second chamber 121b, and third temperature target region 136c heats the reagent in third chamber 121c. A single heating element 123 or a plurality of heating elements 123a-c can be used to obtain the desired temperature. In one embodiment, a higher thermal mass (and hence the thermal inertial) of the heat storage target 125 can be deliberately chosen to facilitate rapid heat transfer to the reagent while the temperature of the heat storage target remains stable. Such a scenario may be advantageous if the reagent is transferred from one chamber/heat storage target to another. In this embodiment a plurality of heat storage targets can be heated to different temperatures corresponding to the hold temperatures required for PCR thermal cycling. Fluidic valves 131a-b, open and close to control the movement of reagent between chambers containing heat storage targets 125a-c. While not shown in the figure, fluidic pumps can be used to create the pressure to move the reagent.

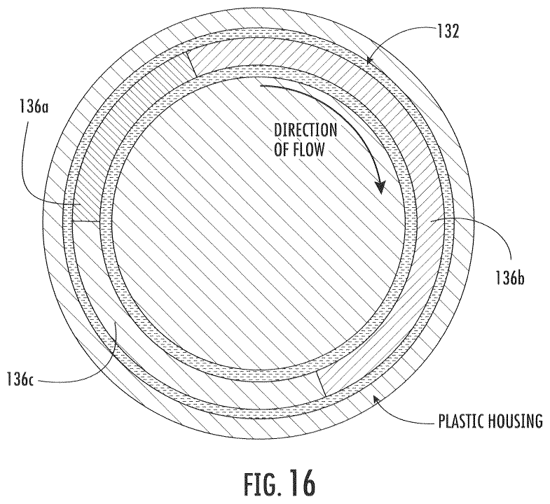

In yet another embodiment shown in FIG. 16, a plurality of temperature target regions 136a-c (e.g., three in this embodiment) are arranged in a continuous (not valved) fluidic channel 132. In this embodiment, the rate of reagent flow through the continuous (not valved) fluidic channel 132 can be constant. Moreover, the length of the individual heat storage targets can be chosen so the time taken for the reagent to traverse the individual target corresponds to the PCR step hold time at the particular temperature. In some embodiments, the number of times the reagent travels (e.g. via pump) around the continuous (not valved) fluidic channel 132 corresponds to the number of PCR cycles. Such a configuration does need valves and a simple fluid pump can be utilized to move the fluid through the targets on the track.

### Example 1: Detection of Chlamydia and Gonorrhea in a Sample Using a Sample-to-Answer Assay Involving PCR Amplification and Lateral Flow Detection

The following is an example of an assay involving PCR amplification and lateral flow analysis of the amplified product carried out using the apparatus for controlling assay processes performed in a sample-to-answer device. The description below provides a practical example of important aspects of the invention discussed above.

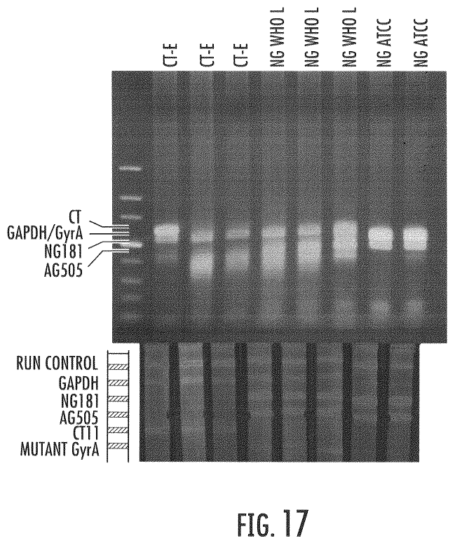

CT and NG cells were spiked in pooled negative vaginal swab samples. CT serovar E spiked at 1.2 IFU/mL, NG WHO-L (ciprofloxacin resistant) at 5 CFU/mL, NG ATCC 430669 (ciprofloxacin sensitive) at 106 CFU/mL. Samples were lysed and purified with charge switch magnetic beads using Applicant's proprietary system. The master mix contains multiplex 5 primers mix to amplify CT, NG, gyrA (ciprofloxacin resistant marker), human GAPDH for sample adequacy control, 1× platinum II PCR buffer (thermos), 5.5 mM MgCl2, 10 U platinum II taq HS DNA polymerase, 120 mM Tris buffer pH 8.8, 0.75× platinum GC enhancer, and 2 μg/μL BSA. Following sample preparation step, amplification was initiated by heating first to 95° C. for 2 minutes to activate the hot start DNA polymerase, then 40 thermal cycling between 95° C. for 15 seconds, and 62.5° C. for 30 seconds. Then amplified products were analyzed by gel electrophoresis. First 4 μl of amplified product was mixed with 1 μL of 5× loading dye, then 3 μL was added to the gel well and run for 13 minutes at 175 V. Following gel analysis, amplified samples were further analyzed by lateral flow strips. Samples were digested by lambda exonuclease enzyme to generate single stranded DNA before being applied to the lateral flow assay. Both gel and lateral flow analysis showed amplification of the corresponding targets. See FIG. 17.

The microfluidic cartridge 116 shown in FIG. 18B includes all the elements necessary to perform the sample-to-answer assay. The cartridge facilitates the sample purification, concentration, amplification and finally the detection of the amplified product. Following lysis, the sample is moved through a filter that separates cellular debris and other PCR inhibitors. The filtered and lysed sample was then passed through the magnetic bead blister where it mixes with the charge switch magnetic bead particles. The sample and magnetic bead mixture then moves to the binding chamber of the cartridge via a debubbler filter which removes air bubbles and prevents them from making its way into the binding chamber. Inside the binding chamber the lysed sample with the magnetic beads mixes with the binding reagents; this changes the pH of the mixture so the DNA in the sample binds to the charge switch magnetic bead particles. The magnetic beads (w DNA bound to them) were then moved into the wash chambers 1 and 2. In these chambers the magnetic beads interact with the wash buffer to remove any PCR inhibitors that might be trapped on the magnetic bead particles. Finally the beads were moved into the amplification chamber 121 where the beads were then suspended in the master mix. The pH of the master mix causes the DNA to elute out of the magnetic bead particles. Thermal cycling for PCR was performed to amplify the DNA. In this example, the amplified product was then moved to the lateral flow strip 133 for detection.

Various methods of detection can be used for detecting the amplified products, including visual detection using pH sensitive dyes or metal-sensitive indicators, electrochemical detection, optical detection using intercalating dyes or fluorescent probes, turbidimetry, lateral flow strip detection. In this example, a lateral flow strip is used to detect the amplified nucleic acids. Biotin and FAM/FITC modified FIP and BIP primers respectively may be used in the LAMP reaction. A sandwich format lateral flow test may be used. The amplified product may be mixed with a dilution or running buffer before lateral flow strip detection. A valve may be present and actuated as part of the assay automation sequence to allow the amplified products to flow on the lateral flow strip. Alternatively, a septum may be pierced to allow the amplified product to flow on the lateral flow strip.

Applicant's instrument embodying the apparatus set forth herein was used to interact with the microfluidic cartridge to automate a sample to answer test. The instrument included the cartridge feed module, blister/reagent dispense module, sample preparation and amplification module, and the detection module. Sample was added into the lysis chamber and the cartridge was partially inserted into the instrument. An RFID tag reader in the instrument read the RFID tag on the cartridge to identify the cartridge type. This is important for the instrument to select the appropriate assay specific sequence file to run.

Assay steps performed by the Applicant's instrument in this example are outlined by the example sequence file below in Table 2 with additional explanation below Table 2.

| | | | | | Hold | H1 | HTR | Feed | Dir | Act | Dir | Plate | Plate | Air |
|---|---|---|---|---|---|---|---|---|---|---|---|---|---|---|
| Stage | Step | Next | Repeat | Degree | Time | ON | 1 SP | Mtr | FM | Mtr | AM | 1 | 2 | Pump |
| 1 | 1 | 0 | 0 | −386 | 0 | 0 | 0 | 1 | 1 | | | | | |
| 2 | 2 | 0 | 0 | −386 | 0 | 0 | 0 | | | 1 | 1 | 1 | | |
| 2 | 3 | 0 | 0 | −386 | 30 | 0 | 0 | | | | | | | 1 |
| 2 | 4 | 0 | 0 | −386 | 0 | 0 | 0 | | | 1 | 1 | | 1 | |
| 2 | 5 | 0 | 0 | −386 | 2 | 0 | 0 | | | | | | | |
| 2 | 6 | 0 | 0 | −50 | 15 | 0 | 0 | | | | | | | |
| 2 | 7 | 0 | 0 | −52 | 3 | 0 | 0 | | | | | | | |
| 2 | 8 | 0 | 0 | −101 | 2 | 0 | 0 | | | | | | | |
| 2 | 9 | 5 | 4 | −95 | 1 | 0 | 0 | | | | | | | |
| 2 | 10 | 0 | 0 | −98 | 8 | 0 | 0 | | | | | | | |
| 2 | 11 | 0 | 0 | −147 | 0 | 0 | 0 | | | | | | | |
| 2 | 12 | 8 | 3 | −149 | 1 | 0 | 0 | | | | | | | |
| 2 | 13 | 0 | 0 | −153 | 0 | 0 | 0 | | | | | | | |
| 2 | 14 | 0 | 0 | −155 | 0 | 0 | 0 | | | | | | | |
| 2 | 15 | 0 | 0 | −157 | 0 | 0 | 0 | | | | | | | |
| 2 | 16 | 0 | 0 | −159 | 0 | 0 | 0 | | | | | | | |
| 2 | 17 | 0 | 0 | −161 | 0 | 0 | 0 | | | | | | | |
| 2 | 18 | 0 | 0 | −163 | 0 | 0 | 0 | | | | | | | |
| 2 | 19 | 0 | 0 | −221 | 5 | 0 | 0 | | | | | | | |
| 2 | 20 | 0 | 0 | −226 | 0 | 0 | 0 | | | | | | | |
| 2 | 21 | 0 | 0 | −230 | 0 | 0 | 0 | | | | | | | |
| 2 | 22 | 0 | 0 | −214 | 3 | 0 | 0 | | | | | | | |
| 2 | 23 | 19 | 4 | −241 | 3 | 0 | 0 | | | | | | | |
| 2 | 24 | 0 | 0 | −230 | 8 | 0 | 0 | | | | | | | |
| 2 | 25 | 0 | 0 | −235 | 8 | 0 | 0 | | | | | | | |
| 2 | 26 | 0 | 0 | −240 | 8 | 0 | 0 | | | | | | | |
| 2 | 27 | 0 | 0 | −173 | 4 | 0 | 0 | | | | | | | |
| 2 | 28 | 0 | 0 | −177 | 4 | 0 | 0 | | | | | | | |
| 2 | 29 | 25 | 4 | −182 | 0 | 0 | 0 | | | | | | | |
| 2 | 30 | 0 | 0 | −175 | 10 | 0 | 0 | | | | | | | |
| 2 | 31 | 0 | 0 | −180 | 0 | 0 | 0 | | | | | | | |
| 2 | 32 | 0 | 0 | −182 | 0 | 0 | 0 | | | | | | | |
| 2 | 33 | 0 | 0 | −184 | 0 | 0 | 0 | | | | | | | |
| 2 | 34 | 0 | 0 | −186 | 0 | 0 | 0 | | | | | | | |
| 2 | 35 | 0 | 0 | −188 | 0 | 0 | 0 | | | | | | | |
| 2 | 36 | 0 | 0 | −250 | 8 | 0 | 0 | | | | | | | |
| 2 | 37 | 0 | 0 | −245 | 0 | 0 | 0 | | | | | | | |
| 2 | 38 | 0 | 0 | −247 | 0 | 0 | 0 | | | | | | | |
| 2 | 39 | 0 | 0 | −232 | 3 | 0 | 0 | | | | | | | |
| 2 | 40 | 36 | 4 | −257 | 3 | 0 | 0 | | | | | | | |
| 2 | 41 | 0 | 0 | −244 | 5 | 0 | 0 | | | | | | | |
| 2 | 42 | 0 | 0 | −252 | 5 | 0 | 0 | | | | | | | |
| 2 | 43 | 0 | 0 | −307 | 0 | 0 | 0 | | | | | | | |
| 2 | 44 | 40 | 3 | −300 | 1 | 0 | 0 | | | | | | | |
| 2 | 45 | 0 | 0 | −303 | 6 | 0 | 0 | | | | | | | |
| 2 | 46 | 0 | 0 | −306 | 4 | 0 | 0 | | | | | | | |
| 2 | 47 | 0 | 0 | −309 | 1 | 0 | 0 | | | | | | | |
| 2 | 48 | 0 | 0 | −311 | 1 | 0 | 0 | | | | | | | |
| 2 | 49 | 0 | 0 | −313 | 1 | 0 | 0 | | | | | | | |
| 2 | 50 | 0 | 0 | −315 | 1 | 0 | 0 | | | | | | | |
| 2 | 51 | 0 | 0 | −317 | 1 | 0 | 0 | | | | | | | |
| 2 | 52 | 0 | 0 | −318 | 1 | 0 | 0 | | | | | | | |
| 2 | 53 | 0 | 0 | −319 | 1 | 0 | 0 | | | | | | | |
| 2 | 54 | 0 | 0 | −321 | 0 | 0 | 0 | | | | | | | |
| 2 | 55 | 0 | 0 | −323 | 0 | 0 | 0 | | | | | | | |
| 2 | 56 | 0 | 0 | −325 | 0 | 0 | 0 | | | | | | | |
| 2 | 57 | 0 | 0 | −327 | 0 | 0 | 0 | | | | | | | |
| 2 | 58 | 0 | 0 | −354 | 0 | 0 | 0 | | | | | | | |
| 2 | 59 | 55 | 3 | −358 | 1 | 0 | 0 | | | | | | | |
| 2 | 60 | 0 | 0 | −360 | 0 | 0 | 0 | | | | | | | |
| 2 | 61 | 57 | 2 | −358 | 0 | 0 | 0 | | | | | | | |
| 2 | 62 | 0 | 0 | −358 | 1 | 0 | 0 | | | | | | | |
| 2 | 63 | 0 | 0 | −358 | 1 | 0 | 0 | | | | | | | |
| 2 | 64 | 0 | 0 | −358 | 1 | 0 | 0 | | | | | | | |
| 2 | 65 | 0 | 0 | −424 | 1 | 1 | 0 | | | | | | | |
| 3 | 66 | 0 | 0 | −386 | 120 | 1 | 95 | | | | | | | |
| 3 | 67 | 0 | 0 | −424 | 0 | 1 | 62.5 | | | | | | | |
| 3 | 68 | 0 | 0 | −386 | 30 | 1 | 62.5 | | | | | | | |
| 3 | 69 | 64 | 39 | −386 | 5 | 1 | 95 | | | | | | | |
| 3 | 70 | 0 | 0 | −424 | 0 | 1 | 62.5 | | | | | | | |
| 3 | 71 | 0 | 0 | −386 | 30 | 1 | 62.5 | | | | | | | |
| 3 | 72 | 0 | 0 | −358 | 0 | 0 | 0 | | | | | | | |
| 5 | 73 | 0 | 0 | −46 | 0 | 0 | 0 | | | | | | | |

The cartridge feed module accepts the microfluidic cartridge 116 and pulls it into the instrument. This module positions the cartridge inside the instrument so the remaining modules on the instrument may interact with the cartridge. In the sequence file step 1: the feed motor turns on and the cartridge 116 is pulled inside the instrument (Dir FM value is +1). See Table 2.

Next, the blister/reagent dispense module (e.g., actuator mechanism 103) activates plate 1 (e.g., 112a) which ruptures the blister seals (e.g., seals for the magnetic bead blister, wash blister 1, wash blister 2 and the amplification buffer blister) and opens the fluidic pathway from the reagent blisters to the respective chambers on the cartridge.

The air pump 137 is now turned ON for 30 s. See FIG. 5B and Table 2. This moves the sample in the lysis chamber to the binding chamber of the cartridge via the sample filter, magnetic bead blister and debubbler filter. Inside the cartridge the following steps happen in this 30 second period:
i. Sample is lysed in the lysis chamber
ii. The lysed sample is then passed through the sample filter to remove cellular debris
iii. The filtered lysate flows through the magnetic bead blister and mixes with the magnetic beads
iv. The lysate and magnetic beads mixture flow over the de-bubbler filter and air bubbles are removed from the sample
v. The mixture makes its way into the binding chamber where it mixes with the binding buffer reagent and the DNA in the sample binds to the magnetic bead particles.

Next, the blister/reagent dispense module (e.g., actuator mechanism 103) actives the plate 2 (e.g., press plate 112b). This plate impinges on the reagent blisters causing them to deform/crush and thereby dispensing the reagents contained within them into the cartridge. This fills the cartridge, i.e., the wash buffer blisters dispense into wash chamber 1 and 2, the amplification blister dispenses into the amplification chamber 121 and the oil blister fills the primary channel of the cartridge. Specifically, Step 2: Actuator motor 110 (Act Mtr) value is set to 1—motor is turned ON, Direction (Dir AM) value is set to 1—travel is towards the cartridge, Plate 1 value is set to 1—clutch 1 is activated that moves plate 1. Step 3: Air Pump 137 value is set to 1 that turns ON the air pump, HoldTime value set to 30 so the air pump is turned ON for 30 seconds. Step 4: Actuator motor (Act Mtr) value is set to 1—motor is turned ON, Direction (Dir AM) value is set to 1—travel is towards the cartridge, Plate 2 112b value is set to 1—clutch 2 104b is activated that moves plate 2 112b. See Table 2, steps 2-4.

At this point, as illustrated in FIG. 18A, the Sample prep and Amplification wheel module (rotating wheel 117) now moves with respect to the cartridge and performs the magnetic bead sample preparation. The sample prep and amplification wheel contains spatially positioned magnets 118 to perform sample purification and concentration. For amplification, the wheel contains a heater element (inductive coil) 123 and a heat sink 128. In this example, the wheel moves with respect to the microfluidic cartridge presenting its magnets to the various chambers of the cartridge.

The following steps (for example, see Table 2, steps 7-12) describe how DNA from 650 μL of the sample is concentrated on the magnetic bead particles, purified by washing steps and finally eluted into 50 μL of solution. FIG. 18A-B illustrates a sample preparation and amplification wheel. FIG. 18B also shows the microfluidic cartridge with the wheel. In this example, the wheel rotates with respect to the microfluidic cartridge to perform magnetic bead based sample concentration and purification.
i. The wheel moves and a magnet on the wheel captures the magnetic bead particles inside the binding chamber and pulls it into the primary channel of the microfluidic cartridge;
ii. As the wheel continues to rotate in a clockwise direction, the magnetic bead particles get deposited on the baffle above wash well 1;
iii. The wheel continues to rotate in a clockwise direction and another magnet on the wheel pulls the magnetic bead particles into wash well 1 where the beads get resuspended into solution and are washed;
iv. As the wheel rotates more, the next magnet collects the magnetic bead particles out of wash well 1 and transfers it above wash well 2;
v. The next magnet brings the magnetic bead particles into the wash well 2 for another wash step;
vi. The wheel continues to rotate and the next magnet on the wheel collects the beads from the wash well 2 and takes it to the amplification chamber;
vii. The next magnet brings the magnetic bead particle into the amplification chamber and resuspends it in the mastermix;
viii. The pH of the mastermix causes the DNA to elute off the magnetic bead particles and into solution of the mastermix.

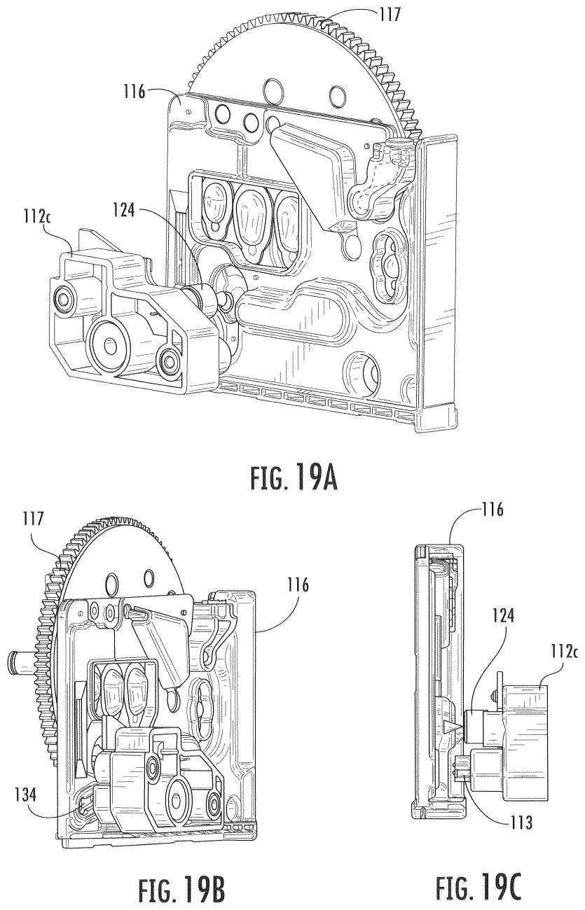

In this example, amplification on the cartridge 116 is performed by thermal cycling of the fluid in the amplification chamber. The heating is performed by induction heating and cooling is done by a heat sink that is cooled with a Thermoelectric cooler. Table 2, steps 64-73 illustrates the PCR thermal cycling sequence file. Generally, the steps are as follows:
i. An actuator wheel 117 (see FIG. 18) rotates and presents the heater element 123 to the amplification chamber of the cartridge. In this example, the heater element is an inductive heating coil that is mounted on the actuator wheel. Inside the amplification chamber 121 of the cartridge, there is a metal target 125 present, and the coil induces heat on the metal target. Since the metal target is in direct contact with the fluid in the amplification chamber, the fluid temperature rapidly rises.
ii. FIG. 19A-C shows the IR sensor 124 positioned in front of the amplification chamber 121. The IR sensor measures the temperature of the fluid inside the amplification chamber.
iii. For the assay used in this example, step 66 (see Table 2) moves the wheel to −386 degrees which presents the inductive coil to the amplification chamber on the cartridge, the heater set point temperature (HTR1SP) value is 95 degrees C. and hold time value is 120 s. So, the inductive coil heats the fluid until the IR sensor measures the temperature of the fluid to reach 95 C. Once at 95 C, the coil retains the heat, so the fluid is held at that temperature for 120 s. Step 66 defines the heat activation step.
iv. Step 67 moves the wheel to −424 degrees which presents the heat sink to the amplification chamber of the cartridge. The heat sink is cooled to 4 degrees C. by a thermoelectric cooler (TEC). The heat sink makes contact with the amplification chamber and pulls heat out of the system, cooling the fluid to 62C.
v. Step 68 moves the wheel back to −386 degrees and presents the inductive coil to the amplification chamber. In this step the inductive coil adds heat into the system to hold the fluid at 62.5 C for 30 seconds. Step 68 defines the annealing/extension step of the PCR.
vi. Step 69 brings the fluid temperature up to 95 C. Step 69 defines the denature step of the PCR.
vii. After completion of step 69, the sequence file indicates the next step is 64 and is repeated 39 times. By performing these steps 40 PCR cycles from 95 C to 65 C with 5 seconds denature step @95 C and 30 seconds of annealing/extension @62.5 C is performed.

Following amplification, the PCR product is moved from the amplification chamber to the lateral flow strip 133 on the cartridge. See FIG. 19A-C illustrating positioning of plate 3 112c of the blister/reagent dispense module in front of the lateral flow strip blister 134. As shown in FIG. 7A, projections 113 on plate 3 (112c) impinge on the lateral flow blister 134 when the plate 3 112c moves forward to the cartridge. The steps are more clearly described below.
i. Plate 3 on the blister/reagent dispense module moves forward to the cartridge and projections on plate 3 impinge on the lateral flow blister. This tears the blister and opens the fluidic pathway from the amplification chamber to the lateral flow strip.
ii. Once the fluidic pathway is opened, the weight of the mineral oil on top of the PCR product provides the pressure head needed to move the PCR product from the amplification chamber to the lateral flow strip via the lateral flow blister.
iii. The lateral flow blister contains an oleophilic/hydrophobic pad. This allows the aqueous PCR product to flow through the blister but traps the mineral oil on the oleophilic pad. Hence only the ˜50 μL of the amplified product makes its way to the lateral flow strip.
iv. On the lateral flow strip the PCR product wicks through the strip and if the target pathogen is present, it binds to the lines on the strip that make them visible.
v. The strip is imaged by a camera and the detection of the pathogen is done by image processing of the Lateral flow strip image. See FIG. 19 which shows a CT11 positive sample.

While the example above describes the use of polymerase chain reaction (PCR) specifically, other NAATs are also contemplated. Recent advances in isothermal amplification assay technologies have simplified the instrumentation requirements for performing NAATs.

### Example 2—Amplification Profiles

### a) Multi-Plex PCR Amplification of CTING in Vaginal Swab Sample

In this example, we spiked CT and NG cells in pooled negative vaginal swab samples. CT serovar E spiked at 1.2 IFU/mL, NG WHO-L (ciprofloxacin resistant) at 5 CFU/mL, NG ATCC 430669 (ciprofloxacin sensitive) at 106 CFU/mL. Samples were lysed and purified with charge switch magnetic beads using NovelDx proprietary system. The master mix contains multiplex 5 primers mix to amplify CT, NG, gyrA (ciprofloxacin resistant marker), human GAPDH for sample adequacy control, 1× platinum II PCR buffer (thermos), 5.5 mM MgCl2, 10 U platinum II taq HS DNA polymerase (Thermo), 120 mM Tris buffer pH 8.8, 0.75× platinum GC enhancer, and 2 μg/μL BSA. Following sample preparation step, amplification was started by heating first to 95° C. for 2 minutes to activate the hot start DNA polymerase, then 40 thermal cycling between 95° C. for 15 seconds, and 62.5° C. for 30 seconds. Then amplified products were analyzed by gel electrophoresis using FlashGel™ System (Lonza). First 4 μL of amplified product was mixed with 1 μL of 5× FlashGel™ loading dye (Lonza), then 3 μL was added to the gel well and run for 13 minutes at 175 V. Following gel analysis, amplified samples were further analyzed by lateral flow strips. Samples were digested by lambda exonuclease enzyme to generate single stranded DNA before being applied to the lateral flow assay. Both gel and lateral flow analysis showed amplification of the corresponding targets.

### b) Rapid RT-PCR Amplification of SARS-CoV-2 Genomic RNA

To demonstrate that NovelDx platform is capable of rapid amplification of a specific target sequence, we used two primers specific for SARS-CoV-2 to amplify 92 and 112 bp targets. We used 2× ready mix One Step PrimeScript™ III RT-PCR Kit (Cat. #RR600B, Takara Bio), enhanced with fast SpeedStar DNA polymerase (TakataBio). The total volume 50 μL of amplification mix consisted of; 14.6 μL water, 25 μL One Step PrimeScript™ III RT-PCR mix, 5 μL primer1 and primer2, (1 μM each), 0.4 μL SpeedStar DNA Polymerase (5 U/μL), 5 μL of gRNA (1000, 100 or 10 copies/reaction). 50 μL was added to the amplification chamber of the cartridge and heated at 55° C. for 2 min for the reverse transcription step to synthesize cDNA heated to 95° C. for 10 seconds to deactivate the RT enzyme and activate the hot start DNA polymerase. Then PCR amplification for 40 cycles between 95° C. for 1 second, and 65° C. for 3 seconds, with a total RT PCR amplification time of around 10.5 minutes. Then amplified products were analyzed by gel electrophoresis using FlashGel™ System (Lonza). First 4 μL of amplified product was mixed with 1 μL of 5× FlashGel™ loading dye (Lonza), then 3 μL was added to the gel well and run for 13 minutes at 175 V. Gel analysis shows two bands below and above the 100 bp DNA marker for 1000 copies and 100 copies of gRNA marker correspond to 92 and 108 bp amplification products. Very faint bands can be seen for the 10 copies, while no bands can be seen for the NTC samples.

### Exemplary Embodiments

1. An apparatus for controlling assay processes in a microfluidic cartridge comprising:
   a single motor;
   a drive belt assembly comprising a single drive belt;
   a clutch assembly comprising one or more clutches configured to rotate about a central axis of rotation, each clutch comprising a drive shaft centrally positioned within each of the one or more clutches on the central axis of rotation, and wherein rotation of said one or more clutches is driven by said drive belt assembly;
   and an actuation mechanism comprising one or more actuation elements mounted to said drive shaft configured to engage the microfluidic cartridge and actuate one or more assay processes.
2. The apparatus of paragraph 1, wherein said one or more actuation elements comprises a press plate.
3. The apparatus of paragraph 1, wherein said microfluidic cartridge comprises one or more reagent filled blisters, a fluidic channel, and one or more wells.
4. The apparatus of paragraph 2, wherein said press plate comprises one or more protrusions configured for physical engagement with said microfluidic cartridge.
5. The apparatus of paragraph 4 wherein said microfluidic cartridge further comprises one or more flow through blisters comprising an inlet valve and an outlet valve and wherein said one or more protrusions comprise a first shape configured to engage and open said inlet valve and outlet valve.
6. The apparatus of paragraph 4 wherein said microfluidic cartridge further comprises one or more reagent filled crush blisters and wherein said one or more protrusions comprise a second shape configured to deform said one or more crush blisters to force said reagent out of said crush blister.
7. The apparatus of paragraph 1, wherein said drive shaft comprises a threaded portion for mounting said one or more actuation elements.
8. The apparatus of paragraph 1 wherein said clutch assembly comprises a plurality of clutches.
9. The apparatus of paragraph 1 wherein said clutch assembly comprises three clutches.
10. The apparatus of paragraph 8 wherein said single motor comprises an axle affixed to only one of said plurality of clutches to power rotation of said drive belt assembly.
11. The apparatus of paragraph 9 wherein said single motor comprises an axle affixed to only one of said plurality of clutches to power rotation of said drive belt assembly.
12. The apparatus of paragraph 3 further comprising a series of spatially arranged permanent magnets positioned proximate said microfluidic cartridge, wherein said microfluidic cartridge further comprises metal particles, and wherein said permanent magnets are configured to move said metal particles through said microfluidic cartridge by magnetic force.
13. The apparatus of paragraph 12 wherein said series of spatially arranged permanent magnets are affixed to a rotating wheel positioned adjacent said microfluidic cartridge.
14. The apparatus of paragraph 1 wherein said one or more clutches are electromagnetic clutches.
15. The apparatus of paragraph 1 wherein said single motor is a stepper motor.
16. The apparatus of paragraph 1 wherein said single motor is a servo motor.
17. The apparatus of paragraph 1 wherein said single motor is a gear motor.
18. The apparatus of paragraph 1 wherein said single motor comprises a single gear and configured to operate at one speed.
19. A method for controlling assay processes in a microfluidic cartridge comprising:
   providing an apparatus configured to be programmed with one or more sequence files corresponding to one or more assays;
   programming said apparatus with one or more sequence files corresponding to said one or more assays;
   wherein said apparatus comprises a single motor; a drive belt assembly comprising a single drive belt; a clutch assembly comprising a plurality of clutches configured to rotate about a central axis of rotation, each clutch comprising a drive shaft centrally positioned within each of the one or more clutches on the central axis of rotation, and wherein rotation of said plurality of clutches is driven by said drive belt assembly; and an actuation mechanism comprising one or more actuation elements mounted to said drive shaft configured to engage the microfluidic cartridge and actuate one or more assay processes;
   inserting the microfluidic cartridge into said apparatus; and
   initiating performance of the assay using the apparatus.
20. The method of paragraph 19 wherein said one or more assay processes comprises polymerase chain reaction.
21. The method of paragraph 19 wherein said one or more assay processes comprises magnetic bead based movement of a target analyte through said microfluidic cartridge.
22. The method of paragraph 19 wherein said one or more assay processes comprises lateral flow strip analysis.
23. An apparatus for controlling assay processes in a microfluidic cartridge comprising a microfluidic cartridge, a temperature sensor, and a rotating wheel that moves with respect to the cartridge, wherein said rotating wheel comprises a heating element, and a heat dissipating element.
24. The apparatus of paragraph 23, wherein said assay is polymerase chain reaction.
25. The apparatus of paragraph 24, wherein said microfluidic cartridge comprises an amplification chamber wherein PCR is performed.
26. The apparatus of paragraph 23, wherein said heating element comprises an inductive coil element.
27. The apparatus of paragraph 23, wherein said heating element is mounted to said rotating wheel.
28. The apparatus of paragraph 23, wherein said heat dissipating element is mounted to said rotating wheel.
29. The apparatus of paragraph 26, wherein said inductive coil element comprises a bifilar coil.
30. The apparatus of paragraph 23, wherein said heat dissipating element comprises a heat sink.
31. The apparatus of paragraph 23, wherein said heat dissipating element comprises a heat sink, a thermoelectric cooler, and/or a heat spreader.
32. The apparatus of paragraph 30, wherein said heat sink is comprised of aluminum, copper (combinations and alloys of the same), carbon-derived materials in combination with aluminum, and/or natural graphite composite materials.
33. The apparatus of paragraph 23, wherein said temperature control unit further comprises a heat storage target.
34. The apparatus of paragraph 33, wherein said heat storage target is positioned inside said amplification chamber.
35. The apparatus of paragraph 33, wherein said heat storage target is comprised of metal.
36. The apparatus of paragraph 33, wherein said heat storage target is positioned on a wall of said amplification chamber, wherein said wall is closest to said heating element.
37. The apparatus of paragraph 35, wherein said metal is aluminum, aluminum alloy, copper, ferrous metals and ferrous alloys, and or combinations thereof.
38. The apparatus of paragraph 33, wherein said heat storage target is comprised of a material comprising predetermined thermal inertia properties.
39. The apparatus of paragraph 38, wherein said heat storage target material comprises high thermal inertia properties.
40. The apparatus of paragraph 23, wherein said temperature sensor comprises an IR temperature sensor.
41. The apparatus of paragraph 25, wherein said heat source and said amplification chamber are not in physical contact.
42. The apparatus of paragraph 25, wherein said temperature sensor and said amplification chamber are not in physical contact.
43. The apparatus of paragraph 23, wherein said temperature control unit is capable of producing heating and cooling rates of said amplification reagent of between about 10° C. per second to about 50° C. per second.
44. The apparatus of paragraph 23 configured to achieve reaction speeds up to about 40 cycles of PCR in under 5 min to 15 mins.
45. The apparatus of paragraph 19 wherein said one or more assay processes is selected from the group lateral flow strip detection, real time optical florescence detection, optical microarray detection, and electrochemical detection.
46. A microfluidic cartridge for use in a sample-to-answer device comprising:
   a microfluidic cartridge comprising a reagent blister and a fluidic channel, wherein said reagent blister comprises a first vessel, a second vessel, a third vessel, an inlet interface for allowing reagent to flow into the blister, an outlet interface for allowing reagent to flow out of the blister and into a fluidic channel, and a rupture bar; and a first plunger, a second plunger, and a third plunger.
47. A sample-to-answer device comprising:
   a microfluidic cartridge comprising a reagent blister and a fluidic channel, wherein said reagent blister comprises a first vessel, a second vessel, a third vessel, an inlet interface for allowing reagent to flow into the blister, an outlet interface for allowing reagent to flow out of the blister and into a fluidic channel, and a rupture bar; and
   a first plunger, a second plunger, and a third plunger.

Based on the above disclosure, it should be apparent to one of ordinary skill in the art that the apparatus for controlling assay processes in a microfluidic cartridge as disclosed above is configured for use in point-of-care sample-to-answer devices or instruments. Thus, the invention described herein also covers point-of-care devices or instruments (or sample-to answer devices or instruments) that include the apparatus for controlling assay processes in a microfluidic cartridge.

### General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

“Nucleic acid” as used herein means a polymeric compound comprising covalently linked subunits called nucleotides. A “nucleotide” is a molecule, or individual unit in a larger nucleic acid molecule, comprising a nucleoside (i.e., a compound comprising a purine or pyrimidine base linked to a sugar, usually ribose or deoxyribose) linked to a phosphate group.

“Polynucleotide” or “oligonucleotide” or “nucleic acid molecule” are used interchangeably herein to mean the phosphate ester polymeric form of ribonucleosides (adenosine, guanosine, uridine or cytidine; “RNA molecules” or simply “RNA”) or deoxyribonucleosides (deoxyadenosine, deoxyguanosine, deoxythymidine, or deoxycytidine; “DNA molecules” or simply “DNA”), or any phosphoester analogs thereof, such as phosphorothioates and thioesters, in either single-stranded or double-stranded form. Polynucleotides comprising RNA, DNA, or RNA/DNA hybrid sequences of any length are possible. Polynucleotides for use in the present invention may be naturally-occurring, synthetic, recombinant, generated ex vivo, or a combination thereof, and may also be purified utilizing any purification methods known in the art. Accordingly, the term “DNA” includes but is not limited to genomic DNA, plasmid DNA, synthetic DNA, semisynthetic DNA, complementary DNA (“cDNA”; DNA synthesized from a messenger RNA template), and recombinant DNA (DNA that has been artificially designed and therefore has undergone a molecular biological manipulation from its natural nucleotide sequence).

“Amplify,” “amplification,” “nucleic acid amplification,” or the like, refers to the production of multiple copies of a nucleic acid template (e.g., a template DNA molecule), or the production of multiple nucleic acid sequence copies that are complementary to the nucleic acid template (e.g., a template DNA molecule).

The terms “top,” “bottom,” “over,” “under,” and “on” are used throughout the description with reference to the relative positions of components of the described devices, such as relative positions of top and bottom substrates within a device. It will be appreciated that the devices are functional regardless of their orientation in space.

“Bead,” with respect to beads on a droplet actuator, means any bead or particle that is capable of interacting with a droplet on or in proximity with a droplet actuator. Beads may be any of a wide variety of shapes, such as spherical, generally spherical, egg shaped, disc shaped, cubical, amorphous and other three dimensional shapes. The bead may, for example, be capable of being subjected to a droplet operation in a droplet on a droplet actuator or otherwise configured with respect to a droplet actuator in a manner which permits a droplet on the droplet actuator to be brought into contact with the bead on the droplet actuator and/or off the droplet actuator. Beads may be provided in a droplet, in a droplet operations gap, or on a droplet operations surface. Beads may be provided in a reservoir that is external to a droplet operations gap or situated apart from a droplet operations surface, and the reservoir may be associated with a flow path that permits a droplet including the beads to be brought into a droplet operations gap or into contact with a droplet operations surface. Beads may be manufactured using a wide variety of materials, including for example, resins, and polymers. The beads may be any suitable size, including for example, microbeads, microparticles, nanobeads and nanoparticles. In some cases, beads are magnetically responsive; in other cases beads are not significantly magnetically responsive. For magnetically responsive beads, the magnetically responsive material may constitute substantially all of a bead, a portion of a bead, or only one component of a bead. The remainder of the bead may include, among other things, polymeric material, coatings, and moieties which permit attachment of an assay reagent. Examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® particles, available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles. Beads may be pre-coupled with a biomolecule or other substance that is able to bind to and form a complex with a biomolecule. Beads may be pre-coupled with an antibody, protein or antigen, DNA/RNA probe or any other molecule with an affinity for a desired target.

“Immobilize” with respect to magnetically responsive beads, means that the beads are substantially restrained in position in a droplet or in filler fluid on a droplet actuator. For example, in one embodiment, immobilized beads are sufficiently restrained in position in a droplet to permit execution of a droplet splitting operation, yielding one droplet with substantially all of the beads and one droplet substantially lacking in the beads.

“Magnetically responsive” means responsive to a magnetic field. “Magnetically responsive beads” include or are composed of magnetically responsive materials. Examples of magnetically responsive materials include paramagnetic materials, ferromagnetic materials, ferrimagnetic materials, and metamagnetic materials. Examples of suitable paramagnetic materials include iron, nickel, and cobalt, as well as metal oxides, such as Fe304, BaFe12019, CoO, NiO, Mn203, Cr203, and CoMnP.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface. In one example, filler fluid can be considered as a film between such liquid and the electrode/array/matrix/surface.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

All publications, patent applications, patents, and other references (including references to specific commercially available products or product lines) mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

What is claimed is:

1-47. (canceled)

48. An apparatus for controlling assay processes in a microfluidic cartridge comprising:
a single motor;
a drive belt assembly comprising a single drive belt;
a clutch assembly comprising one or more clutches configured to rotate about a central axis of rotation, each clutch comprising a drive shaft centrally positioned within each of the one or more clutches on the central axis of rotation, and wherein rotation of said one or more clutches is driven by said drive belt assembly;
and an actuation mechanism comprising one or more actuation elements mounted to said drive shaft configured to engage the microfluidic cartridge and actuate one or more assay processes.

49. The apparatus of claim 48, wherein said one or more actuation elements comprises a press plate comprising one or more protrusions configured for physical engagement with said microfluidic cartridge and wherein said microfluidic cartridge comprises one or more reagent filled blisters, a fluidic channel, and one or more wells.

50. The apparatus of claim 49 wherein said microfluidic cartridge further comprises one or more flow through blisters comprising an inlet valve and an outlet valve and wherein said one or more protrusions comprise a first shape configured to engage and open said inlet valve and outlet valve.

51. The apparatus of claim 49 wherein said one or more protrusions comprise a second shape configured to deform said one or more reagent filled blisters to force said reagent out of said reagent filled blister.

52. The apparatus of claim 48, wherein said drive shaft comprises a threaded portion for mounting said one or more actuation elements.

53. The apparatus of claim 48 wherein said clutch assembly comprises a plurality of clutches powered by said single motor.

54. The apparatus of claim 53 wherein said single motor comprises an axle affixed to only one of said plurality of clutches to power rotation of said drive belt assembly and an axle affixed to only one of said plurality of clutches to power rotation of said drive belt assembly.

55. The apparatus of claim 49 further comprising a series of spatially arranged permanent magnets positioned proximate said microfluidic cartridge and affixed to a rotating wheel positioned adjacent said microfluidic cartridge, wherein said microfluidic cartridge further comprises metal particles, and wherein said permanent magnets are configured to move said metal particles through said microfluidic cartridge by magnetic force.

56. The apparatus of claim 48 wherein said one or more clutches are electromagnetic clutches.

57. The apparatus of claim 48 wherein said single motor is a stepper motor, a servo motor, or a gear motor.

58. The apparatus of claim 48 wherein said single motor comprises a single gear and configured to operate at one speed.

59. A method for controlling assay processes in a microfluidic cartridge comprising:
providing an apparatus configured to be programmed with one or more sequence files corresponding to one or more assays;
programming said apparatus with one or more sequence files corresponding to said one or more assays;
wherein said apparatus comprises a single motor; a drive belt assembly comprising a single drive belt; a clutch assembly comprising a plurality of clutches configured to rotate about a central axis of rotation, each clutch comprising a drive shaft centrally positioned within each of the one or more clutches on the central axis of rotation, and wherein rotation of said plurality of clutches is driven by said drive belt assembly; and an actuation mechanism comprising one or more actuation elements mounted to said drive shaft configured to engage the microfluidic cartridge and actuate one or more assay processes;
inserting the microfluidic cartridge into said apparatus; and
initiating performance of the assay using the apparatus.

60. The method of claim 59 wherein said one or more assay processes comprises polymerase chain reaction.

61. The method of claim 59 wherein said one or more assay processes comprises magnetic bead based movement of a target analyte through said microfluidic cartridge.

62. The method of claim 59 wherein said one or more assay processes comprises lateral flow strip analysis.

63. An apparatus for controlling assay processes in a microfluidic cartridge comprising a microfluidic cartridge, a temperature sensor, and a rotating wheel that moves with respect to the cartridge, wherein said rotating wheel comprises a heating element, and a heat dissipating element, and wherein said assay is polymerase chain reaction.

64. The apparatus of claim 63, wherein said heating element comprises an inductive coil element.

65. The apparatus of claim 63, wherein said heating element and said heat dissipating element is mounted to said rotating wheel.

66. The apparatus of claim 64, wherein said inductive coil element comprises a bifilar coil.

67. The apparatus of claim 63, wherein said heat dissipating element comprises a heat sink, a thermoelectric cooler, and/or a heat spreader.

68. The apparatus of claim 67, wherein said heat sink is comprised of aluminum, copper (combinations and alloys of the same), carbon-derived materials in combination with aluminum, and/or natural graphite composite materials.

69. The apparatus of claim 63, further comprising a temperature control unit comprising a heat storage target positioned inside an amplification chamber within said microfluidic cartridge.

70. The apparatus of claim 69, wherein said heat storage target is positioned on a wall of said amplification chamber, wherein said wall is closest to said heating element and is comprised of metal.

71. The apparatus of claim 70, wherein said metal is aluminum, aluminum alloy, copper, ferrous metals and ferrous alloys, and or combinations thereof.

72. The apparatus of claim 69, wherein said heat storage target is comprised of a material comprising predetermined thermal inertia properties.

73. The apparatus of claim 72, wherein said heat storage target material comprises high thermal inertia properties.

74. The apparatus of claim 63, wherein said temperature sensor comprises an IR temperature sensor.

75. The apparatus of claim 63, wherein said microfluidic cartridge comprises an amplification chamber and wherein PCR is performed heating element and said amplification chamber are not in physical contact.

76. The apparatus of claim 75, wherein said temperature sensor and said amplification chamber are not in physical contact.

77. The apparatus of claim 63, wherein said temperature control unit is capable of producing heating and cooling rates of said amplification reagent of between about 10° C. per second to about 50° C. per second.

78. The apparatus of claim 63 configured to achieve reaction speeds up to about 40 cycles of PCR in under 5 min to 15 mins.

79. The apparatus of claim 59 wherein said one or more assay processes is selected from the group lateral flow strip detection, real time optical florescence detection, optical microarray detection, and electrochemical detection.
